(19) United States
(12) Patent Application Publication
(10) Pub. No.: US 2009/0240262 A1
(43) Pub. Date: Sep. 24, 2009

(54) Device for affixing prosthesis to a vessel

(75) Inventors: Edward G. SHIFRIN, Raanana (IL); Mordehi D. SHVARTSMAN, Haifa (IL); Mark A. UMANSKY, Haifa (IL); Ralf KOLVENBACH, Dusseldorf (DE); Ilya SITNITSKY, Nahariya (IL)

Correspondence Address: MINTZ LEVIN COHN FERRIS GLOVSKY & POPEO, ONE FINANCIAL CENTER, BOSTON, MA, 02111, US

(73) Assignee: ES Vascular, Ltd., Haifa Bay (IL)

(21) Appl. No.: 12/077,819

(22) Filed: Mar. 20, 2008

(51) Int. Cl. A61B 17/10; A61F 2/06

(52) U.S. Cl. 606/139; 623/1.13; 623/1.11

(57) ABSTRACT

Embodiments of the present invention are directed to devices, systems and methods (both in operation of the devices and systems, and methods of treatment on a mammal) for affixing a prosthesis, e.g. graft to a first end and to a second end of a vessel is described. Such embodiments include a device which may comprise a rear handle portion, a frontal working portion and an intermediate barrel portion. The working portion may be loaded with a first plurality of staples and with a second plurality of staples. The barrel portion is preferable adapted to carry the graft thereon and the device is preferably provided with a pushing rod which may be displaceable along the barrel portion so as to cause protrusion of the first and the second plurality of staples towards the first end and towards the second end of the vessel respectively.

### FIELD OF THE INVENTION

The present invention is generally within the field of medicine, particularly pertaining to vascular surgeries during which a prosthesis is introduced into a vessel and secured at the internal vessel wall.

More specifically, the present invention refers to devices and methods used during surgical operations associated with treating large vessels, e.g. for treating the superficial femoral artery by inserting a prosthesis, e.g. graft into the artery without opening the abdominal and thoracic cavities. Such surgical operations may be used instead of bypass surgery. It should be borne in mind, however, that the present invention is not limited merely to affixing a prosthesis to the internal wall of the superficial femoral artery. The present invention also can be used in surgical operations associated with introducing other prostheses in other vessels, for example the brachial artery or any other large vessels which have an internal diameter of at least 5 mm, preferably 6-8 mm.

### BACKGROUND OF THE INVENTION

Some surgical devices for affixing a prosthesis to the internal wall of a body lumen have been disclosed in the art.

For example, J. Parodi discloses systems and methods for securing a single stitch inside the vascular lumen in U.S. Pat. No. 6,336,933, EP 1308131 A1, and US Pat. Appl. 2003/0023248.

In U.S. Pat. No. 6,592,593 and in WO 00/16701, J. Parodi discloses an applicator, which can be set up inside a vessel for affixing a prosthesis to the vessel wall.

In US Pat. Appl. 2002/0177862 and WO 00/64357, E. Arany et al. disclose a device for the endovascular affixing of a prosthesis using at least one stapler. The device has a chamber loadable with additional staplers.

The known methods and systems of delivery and affixing an endovascular prostheses usually employ two separate systems, which fulfill different tasks. For example, there is a system which delivers the prosthesis into the vessel, and there is a separate system for affixing the prosthesis. Additional methods and devices based on this principle are disclosed in the following references: U.S. Pat. Nos. 5,443,477, 5,507,769, 5,591,196, 5,618,300, 5,695,517, 6,039,749, 6,168,610, 6,575,994, WO 95/21593, 96/11648, EP 1290989.

U.S. Pat. No. 7,125,412 discloses an apparatus that allows affixing a prosthesis to the aorta wall using staples. During a surgical operation, an incision is cut in the aorta wall, the apparatus is inserted into the aorta, and the prosthesis is affixed to the aorta wall. Unfortunately, this apparatus can be employed only in the course of such a surgical operation. Furthermore, this apparatus requires the use of an external support means for reliably securing the staple edges. Otherwise, without the external support, it is not possible to reliably secure a prosthesis in vessels having thin walls with thickness of 0.2-0.4 mm.

There also exists an alternative method of vessel treatment known as an “endovascular prosthesis,” whereby the endo-prosthesis (e.g., a woven polyester tube with a reinforcing inner metal frame) is set into a vessel, e.g., into an aneurism. This method does not require incision of the vessel or the tissue, which surrounds the vessel. This method may be used in patients for whom a surgical operation may be not recommended for any reason as well as in patients who prefer not to go through a surgical operation. During the endovascular prosthesis treatment, a stent-graft (endo-prosthesis) is used to strengthen a weak vessel wall and to prevent the vessel's rupture. The endo-prosthesis is brought into the vessel using a dedicated delivering catheter (e.g., a long, tube-like device). Implanting of the endo-prosthesis is carried out under local/epidural anesthesia or narcosis. To carry out the procedure of endovascular prosthesis, only two small cuts are made at the upper part of each hip.

Endovascular prosthesis treatment is an efficient alternative in the treatment of aneurisms of large vessels such as those which may form on the abdominal part of the aorta. This method allows:
the reduction or avoidance of narcosis and lung ventilation;
the reduction or avoidance of damage to the blood circulation in vital organs and in lower extremities;
the reduction or avoidance of complications, which may occur after an open surgery;
the reduction of hospitalization and rehabilitation time;
the reduction in the loss of blood.

The device described in U.S. patent application 60/908,787, filed on 29 Mar. 2007, is capable of affixing stent-grafts to the aorta wall after the stent-graft is inserted into the aorta during an endovascular procedure. U.S. patent application 60/908,787 is incorporated herein by reference in its entirety.

The device described in U.S. patent application 60/908,78 comprises a body with a longitudinally displaceable push member as well as a single working head preloaded with staples for affixing the stent-graft and is fitted with a plurality of pivoting wings that carry the staples. The wings may be swiveled when the push member is longitudinally displaced in a distal direction such that upon swiveling, the wings approach the internal surface of the stent-graft and stretch it in a radial direction. The working head is provided with pivoting levers that also may be swiveled by the push member at the end of its distal displacement such that the staples are forced by the levers to exit out from the working head and to protrude radially through the stent-graft wall, through the vessel wall, and eventually to affix one end of the stent-graft to the vessel wall. During radial protrusion, the staples meet stretched walls of the stent-graft that function as support. This support allows a reliable securing of the stent-grafts within a vessel.

Unfortunately, the above device is suitable only for securing prostheses that are brought in place by a dedicated catheter.

Prostheses, such as grafts, which are used to shunt large vessels, can't be brought into place by a dedicated catheter and require use of the same instrument to deploy the prosthesis as that which secures the prosthesis at the vessel wall.

Furthermore, since in large vessels the graft should be secured at the two ends of the graft, the above-mentioned device is not suitable since its working head has only a limited amount of preloaded staples. This amount is sufficient for securing only one graft end. For securing the opposite end the endovascular prosthesis procedure should be repeated with a fresh device, which is reloaded with new portion of staples, which is not always possible.

Thus, it can be stated that despite the existence of many attempts to devise a device for affixing a prosthesis to a vessel, there still exists a need for a new and improved solution.

### SUMMARY AND OBJECTS OF THE INVENTION

In some embodiments of the present invention, a device for affixing a prosthesis (e.g., graft) to a vessel is provided, which is based on the operating principle of devices used for securing stent-grafts in small vessels, but at the same time is preferably capable of securing grafts in large vessels, having diameter of at least about 5 mm (for example).

An object of some of the embodiments of the invention is to provide a device which will be suitable for affixing two opposite ends of a graft to a vessel by the same device during a single endovascular prosthesis procedure.

Another object of some of the embodiments of the invention is to provide a device which enables reliable, fast and convenient affixing of a graft to a large vessel.

Yet another object of some of the embodiments of the present invention is to provide a device which is provisionally connectable to both graft ends so as to enable bringing the graft in place together with the device and then affixing one end of the graft. After completing this stage, the opposite end of the graft is disconnected from the device and the device is displaced along the vessel to the opposite end of the graft, and then this end of the graft is affixed to the vessel.

### BRIEF DESCRIPTION OF THE DRAWINGS

FIG. 1 is a general isometric view of a device of the invention.

FIG. 2 is a general isometric view of a device of the invention carrying a graft.

FIG. 3 shows how a device of the invention is manually operated when a handle portion is gripped by an operator's hand.

FIG. 4 shows schematically the human leg and femoral artery, into which a graft can be inserted and affixed.

FIG. 5 shows how a device of the invention is brought towards the artery and is ready for inserting in the artery through an incision made at a proximal end of the hip.

FIG. 6 shows a device of the invention after it has been introduced into the artery with its working portion brought to a distal end of the artery.

FIG. 7 is an enlarged view of the distal end of the artery with a working portion of the device inside.

FIG. 8 shows how a device of the invention is being operated when a working portion is at the distal end of the artery.

FIG. 9 is an enlarged view of a distal end of the device of the invention when frontal staples are being pushed by a working portion.

FIG. 10 shows evacuation of a working portion from the artery after the distal end of the graft has been affixed and the proximal end of the graft has been disconnected.

FIG. 11 is an enlarged view of the proximal end of the artery with a working portion inside.

FIG. 12 is an enlarged view of the proximal end of the artery with rear staples protruding into the wall of the artery.

FIG. 13 shows evacuation of a working portion from the vessel.

FIG. 14 is an exploded view of a device of the invention.

FIG. 15 is a side view of a working portion and a rear handle portion.

FIG. 16 is an exploded view of a working portion with front and rear groups of pivoting elements.

FIG. 17 is a cross-section A-A of FIG. 15.

FIG. 18 is a cross-section B-B of FIG. 15.

FIG. 19 is an enlarged isometric cross-section view of a working portion showing a pushing rod.

FIG. 20 is an enlarged view of a working portion

FIG. 21 shows a pushing rod.

FIG. 22 shows a forward head of a pushing rod.

FIG. 23 shows a pushing rod protracting into a working portion.

FIG. 24 shows an initial stage of actuating a front group of pivoting elements.

FIG. 25 shows a final stage of actuating a front group of pivoting elements.

FIG. 26 shows a working portion in a second working stage when a pushing rod has been retracted proximally and rotated.

FIG. 27 shows an initial stage of actuating rear groups of pivoting elements.

FIG. 28 shows a final stage of actuating frontal groups of pivoting elements.

FIG. 29 is a partially cross-sectioned isometric view of a handle portion.

FIG. 30 is an isometric view of an external guiding bushing.

FIG. 31 is an isometric view of a collet member.

FIG. 32 is an isometric view of a inner guiding bushing.

FIG. 33 shows a rear handle portion being locked by a latch.

FIG. 34 shows a rear handle portion in a first working position.

FIG. 35 shows a rear handle portion in a second working position.

FIG. 36 shows a rear handle portion after an external bushing has been displaced distally.

FIG. 37 shows a rear handle portion in a position when the pushing rod is retracted and turned by the torsion spring.

FIG. 38 shows a handle portion being ready for protracting a pushing rod and actuating a rear groups of pivoting elements.

FIG. 39 shows a handle portion during actuating a rear group of pivoting elements.

### DETAILED DESCRIPTION

Referring to the Figures, it will be discussed first how a device according to some embodiments of the present invention are used in practice for affixing two ends of a graft to a vessel.

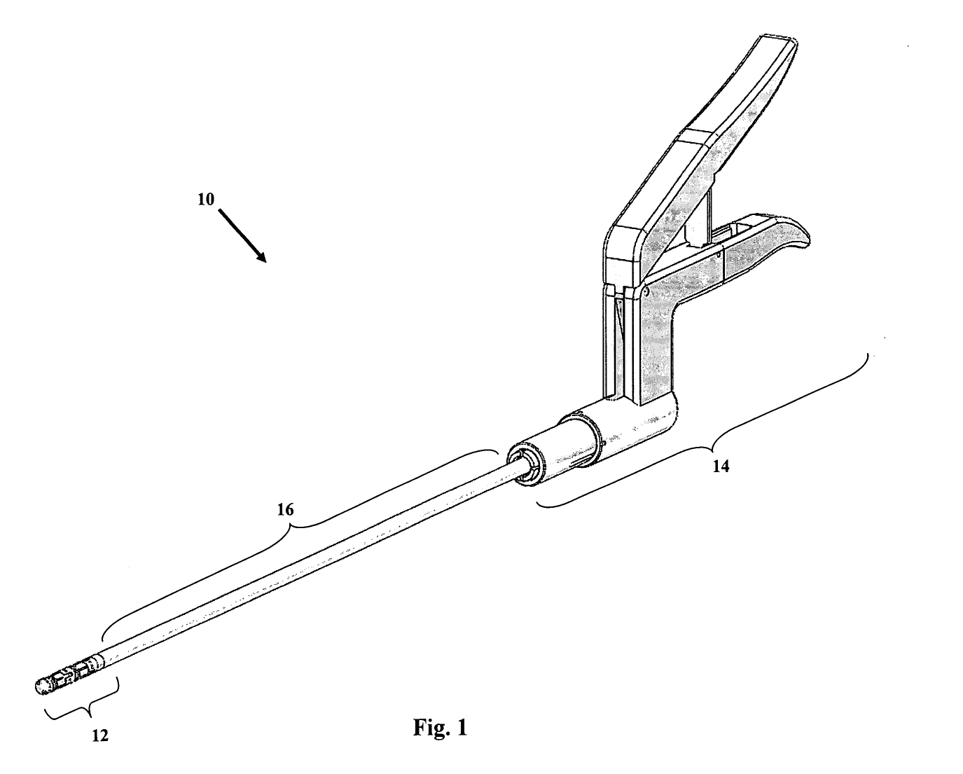

With reference to FIG. 1 a device 10 according to some embodiments of the invention is shown comprising a frontal working portion 12, a rear handle portion 14, and an intermediate barrel portion 16. Upon manually operating the handle portion, the working portion may be activated and consecutively affixes two opposite ends of a graft to an internal wall of a vessel. Before using the device, the graft is preferably put on the barrel portion such that the graft preferably extends along both the barrel portion and the working portion and covers them. In practice, the graft may be a pliable tube made of a plastic material having good compatibility with the bodily tissues. The elastic properties of the graft material preferably ensure that when the graft is put on the barrel portion, the graft adjoins the barrel portion preferably without (however) exerting pressure on it. In some embodiments, the graft includes an outside diameter of at least about 5 mm and a wall thickness of about 0.2 mm.

An example of a suitable plastic material for grafts may be Teflon or PTFE (PolyThetraFluorEthylene). The outside diameter of grafts, which can be reliably affixed to the femoral artery (for example) by a device according to some embodiments of the present invention, may be between about 6-8 mm. The length dimension of the graft depends on the vessel to which the graft has to be affixed. In practice, it may vary between about 300 and 500 mm. Accordingly, the length of the barrel portion may be at least 300 mm, according to some embodiments.

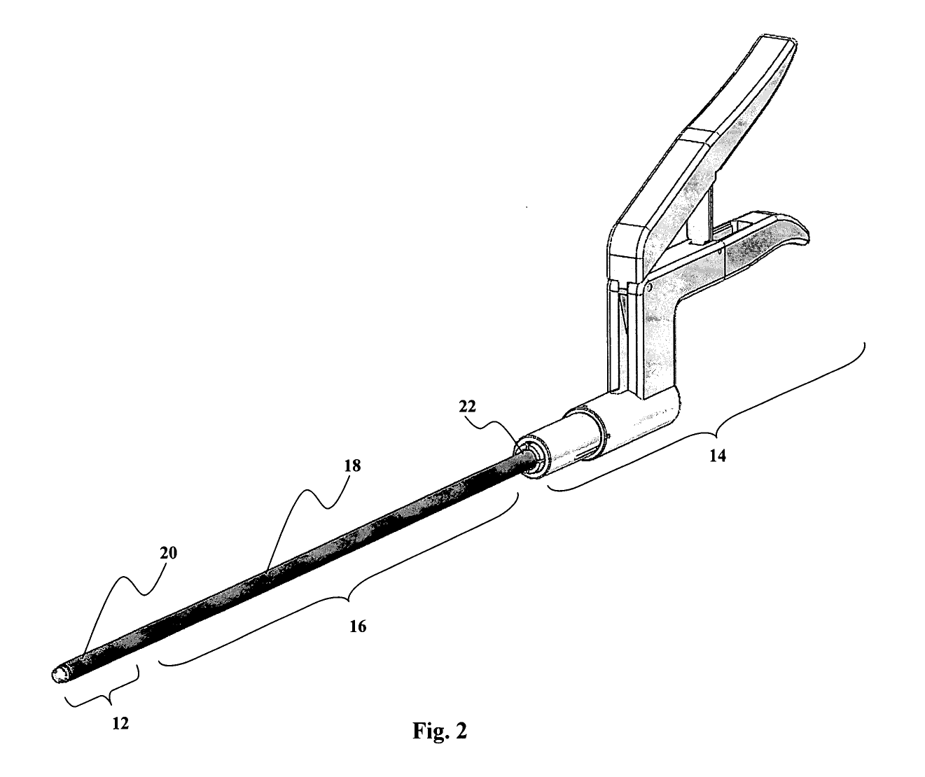

According to some embodiments, when using the device, the barrel portion may be brought within a vessel, where the graft has to be affixed. In FIG. 1, the device is shown without the graft; and in FIG. 2, the device is shown with the barrel portion carrying a graft 18 put thereon. Two opposite ends of the graft may be provisionally connected to the device such that a proximal end 20 of the graft may be provisionally connected to the handle portion 14, and a distal end 22 of the graft is provisionally connected to the working portion 12. It will be discussed below that upon affixing the distal end of the graft, the proximal end may no longer provisionally connect to the handle portion; and thus relative displacement of the barrel portion along the vessel becomes possible.

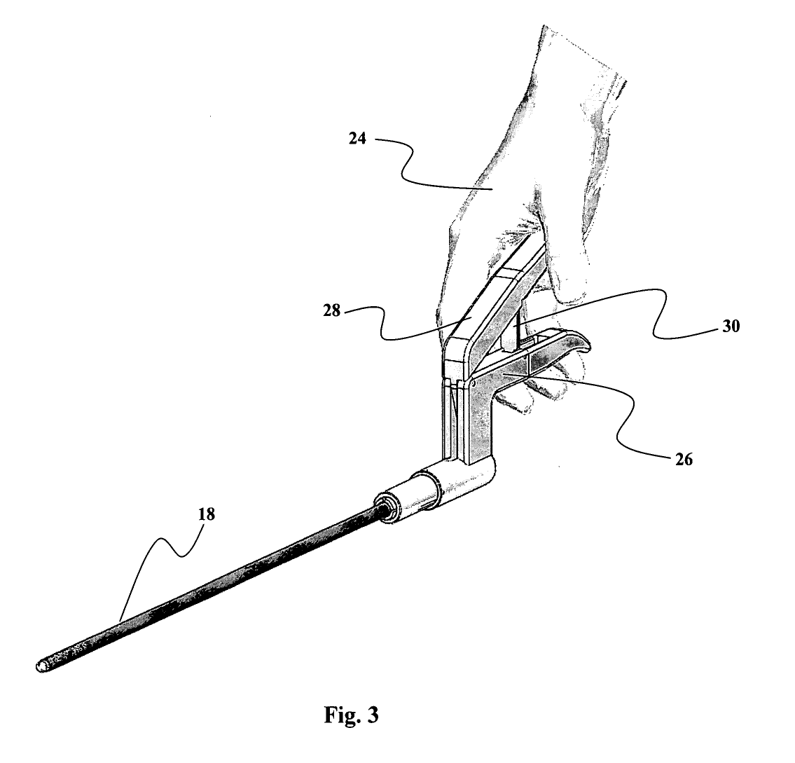

With reference to FIG. 3, it is shown how an operator's hand 24 can hold the handle portion. The handle portion is provided with two opposite handles 26, 28 which can be pressed to approach each other to operate the device and to activate the working portion. To prevent inadvertent pressing of the handles and undesirable activation of the working portion, there is provided a removable swiveling latch 30, which locks the handles when brought into an upright position.

In FIG. 3, the device is shown as ready for operation, except the handle portion is locked by the latch 30 and can be unlocked to bring one handle close to the other handle.

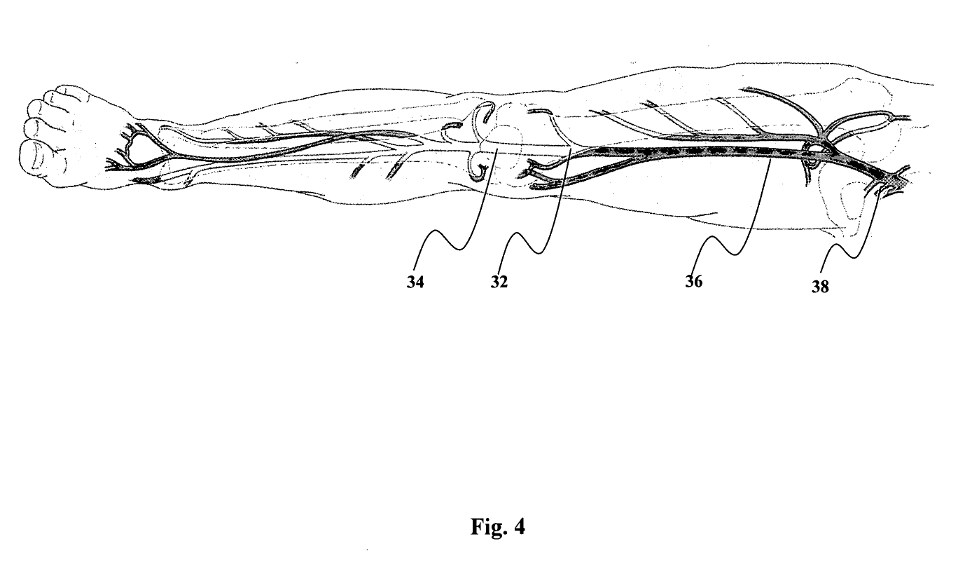

Referring to FIG. 4, a leg of a patient is shown with a femoral artery 32 extending along a hip portion of the leg. When an endovascular prosthesis procedure is required, the device according to some embodiments of the invention allows introduction of a graft into the femoral artery (for example) and affixing the graft preferably at two ends of the internal wall of the artery. This is preferably affected first at a distal end 34 of the artery and then at a proximal end 36 of the artery. The device according to some embodiments allows delivering the graft inside the femoral artery by introducing the barrel portion where the graft surrounds the barrel portion. The barrel portion may be introduced through a transversal incision 38 cut in the hip portion near the proximal end 36.

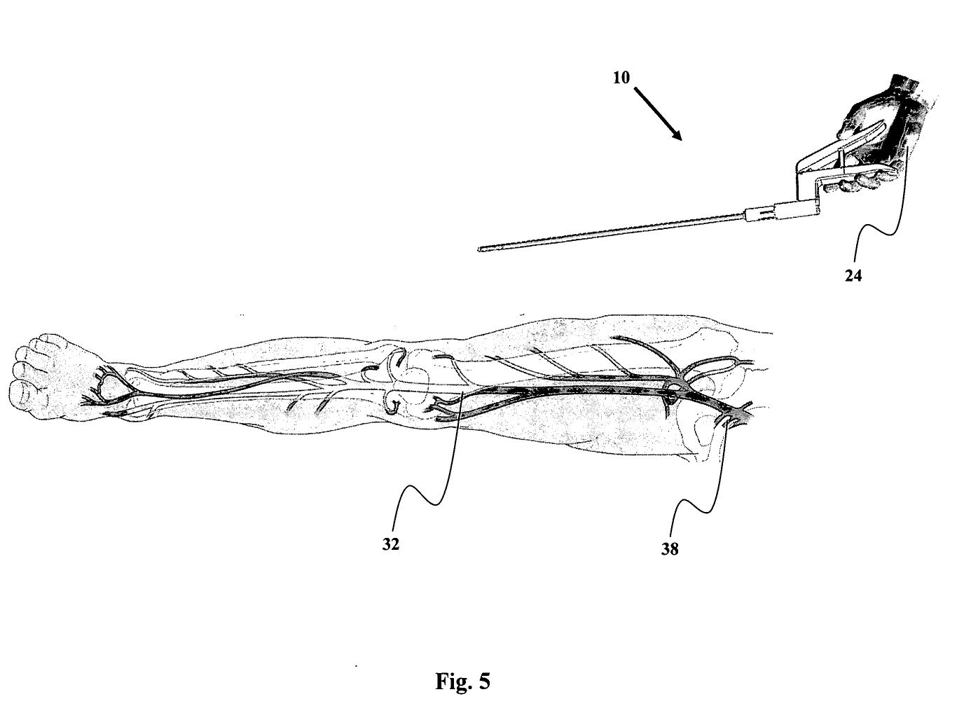

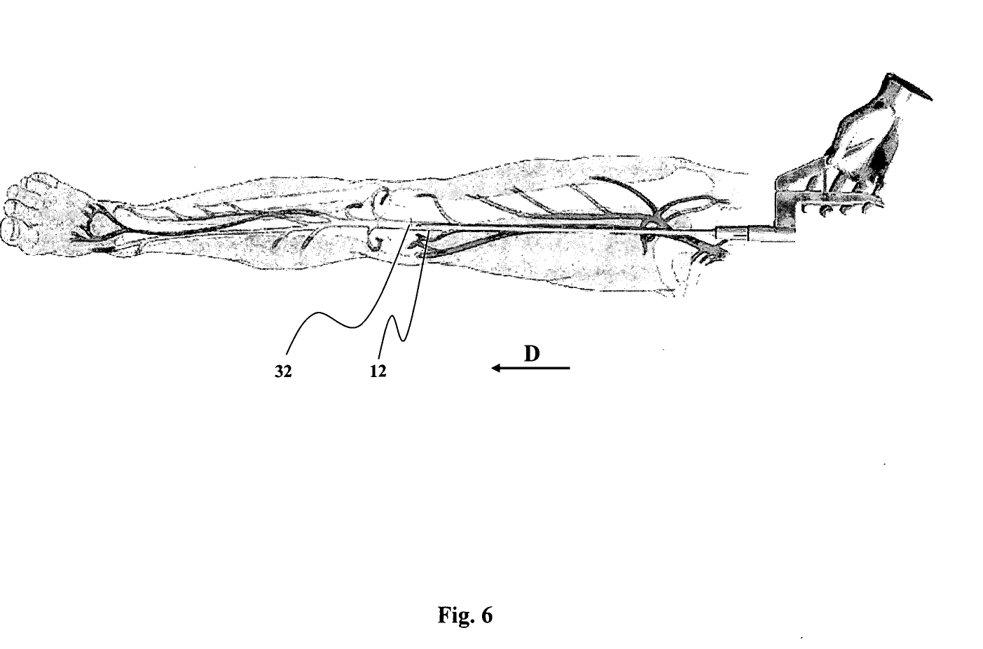

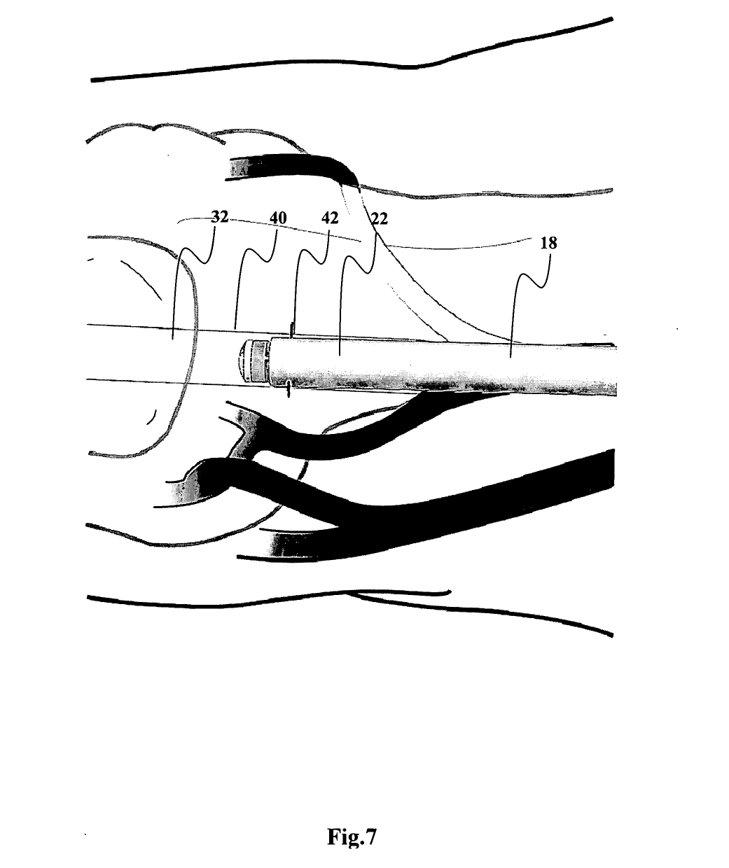

FIG. 5 shows the device gripped by an operator's hand and brought towards the leg to carry out the endovascular prosthesis procedure according to some embodiments. FIG. 6 shows the barrel portion after it has been protracted in a distal direction, designated by an arrow D. As shown, the barrel portion has been protracted distally within the artery until the working portion 12 of the device approaches the distal end 32 of the artery where the distal end of the graft preferably is affixed. The device is shown ready for affixing the graft. According to some embodiments, the following exemplary method may be used to affix a graft using the device. The operator unlocks the handle portion by swiveling the latch and pressing the handles so as to actuate the working portion. As is further described in more detail, this results in affixing the distal end of the graft to a wall 40 of the artery at its distal end. FIG. 7 shows the distal end 22 of the graft located within the artery and being delivered there by the barrel portion. The graft is provisionally connected to the working portion by a surgical fastener, e.g. staple 42 slightly protruding into wall 40. As discussed below, the working portion may be loaded with two groups of staples, which which may comprise frontal groups and rear groups (for example). The staple 42 shown in FIG. 7 belongs to one of the frontal groups of staples. The frontal group of staples preferably affixes the distal end of the graft and the rear groups of staples preferably affixes the proximal end of the graft.

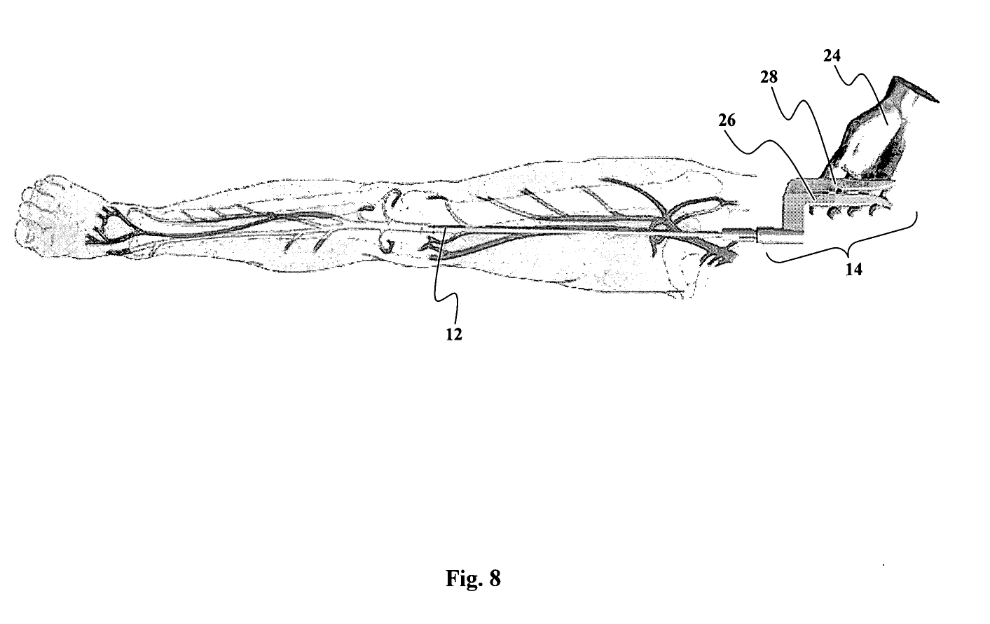

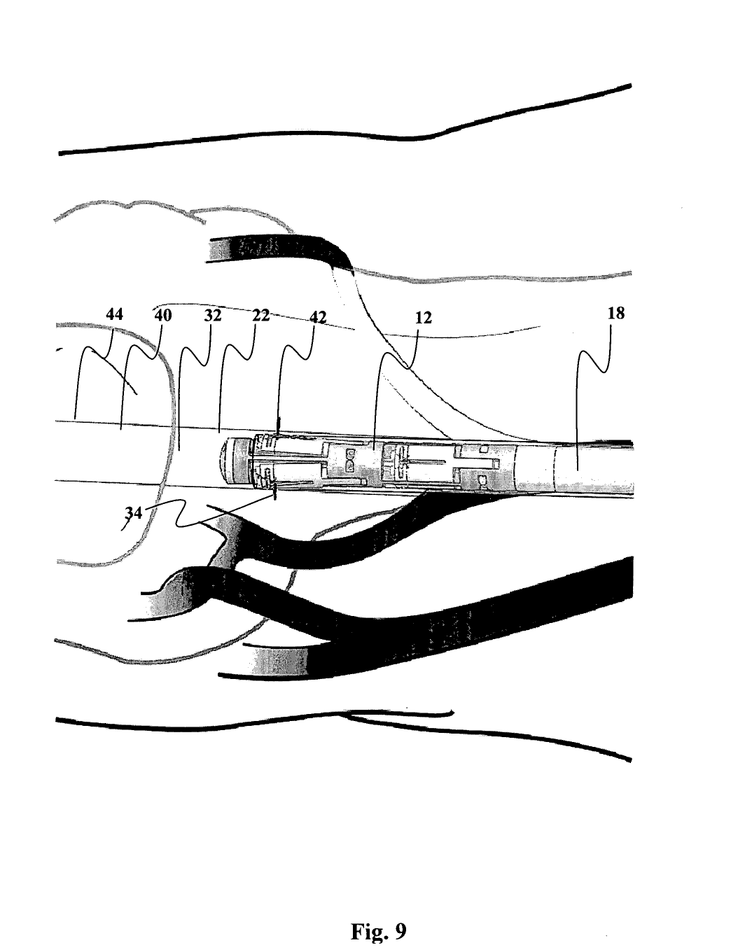

Referring to FIG. 8, the handle portion 14 may be operated by the operator's hand 24 which presses the handles 26,28 so as to activate the working portion 12. As seen in FIG. 9, upon activating the working portion, the staple 42 of the frontal group, and preferably along with the other staples (not shown), protrude out of the working portion until the staples pierce the wall 40 of the graft and a wall 44 of the artery. The staples may then secure the distal end 32 of the graft 18 at the distal end 34 of the artery 32. It should be appreciated that, according to some embodiments, upon the staples protruding, the staples escape the working portion; releasing the distal end of the graft from the working portion. Upon release of the working portion from the distal end of the graft, the handle portion is also preferably disconnected from the proximal end of the graft; and at this point the barrel portion is free to be displaced proximally from the artery, leaving the graft inside the artery with the its distal end affixed to the distal end of the artery. The mechanism responsible for protrusion of the frontal group of staples is discussed in more detail below.

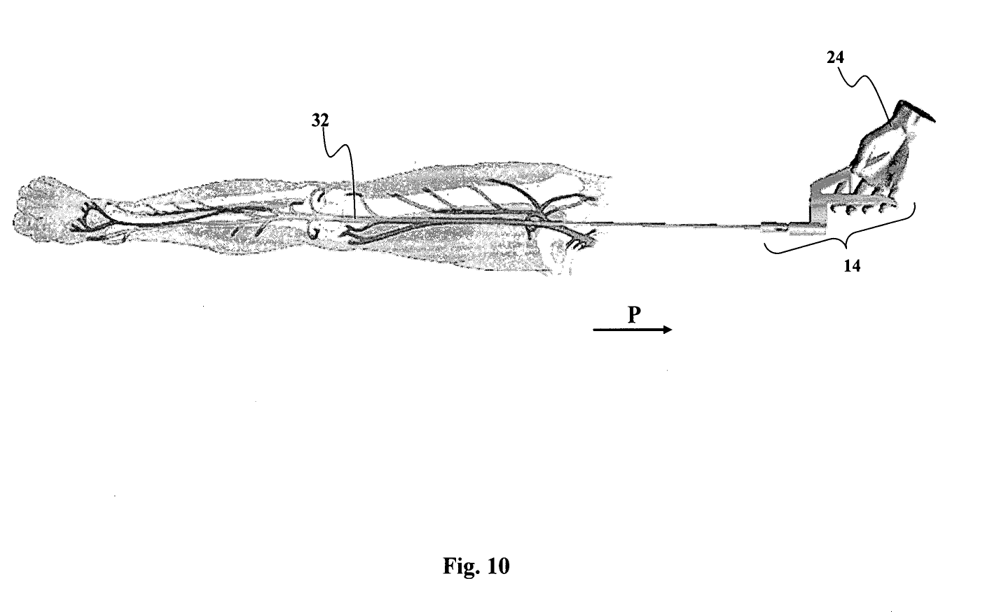

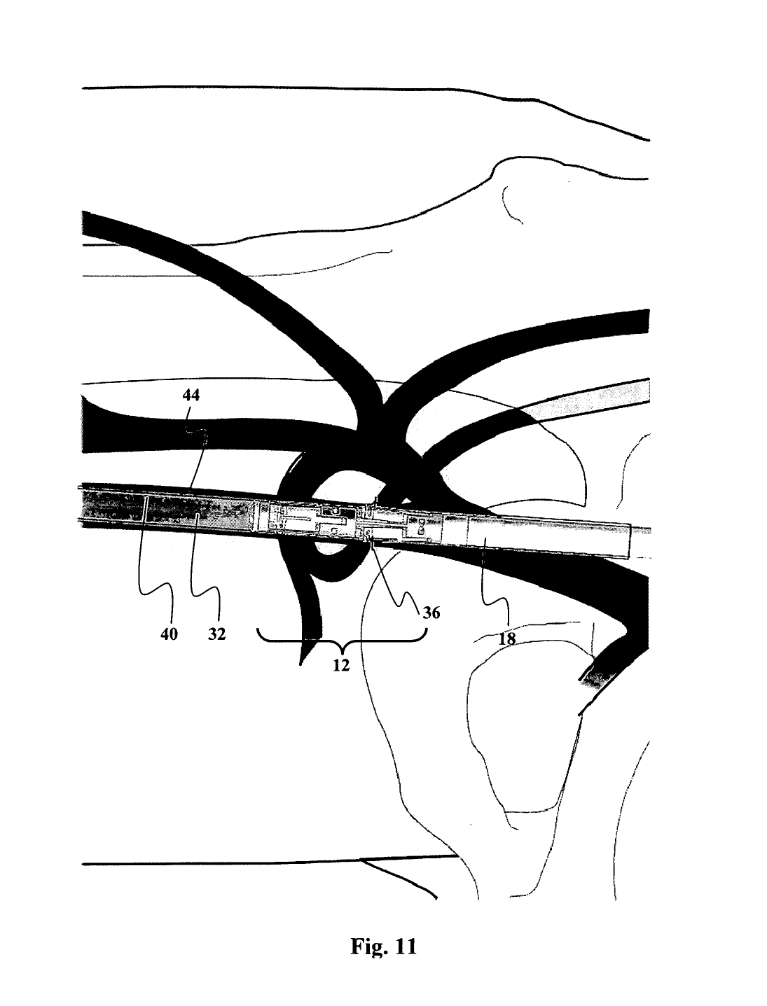

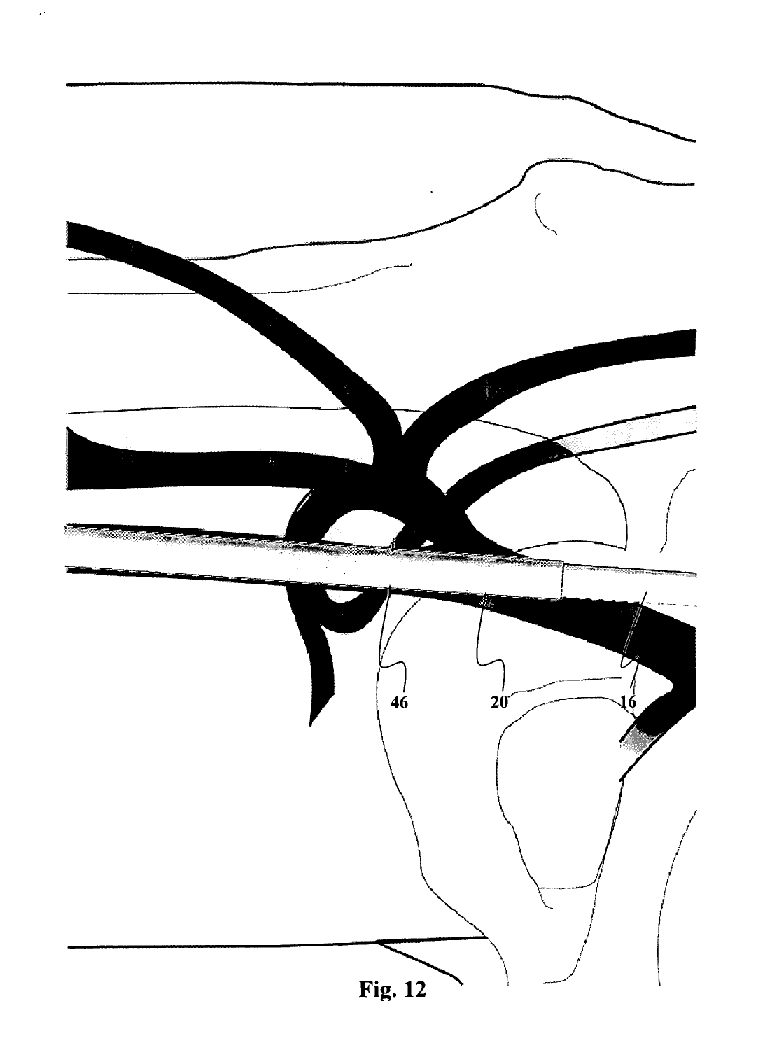

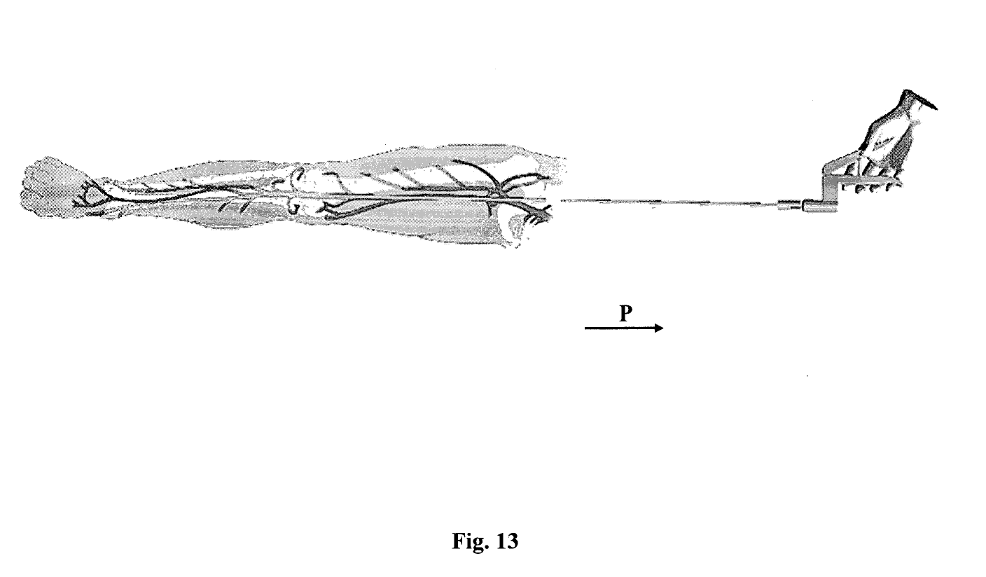

Referring to FIG. 10, the working portion 12 of the device is shown retracted from the artery 32 in a proximal direction, designated by an arrow P. The operator has not yet operated the handle portion, which is still in the operator's hand 24. As soon as the barrel portion is retracted to the extent that the working portion is brought to the proximal end 20 of the graft 18, the handle portion may be operated by the operator's hand to activate the working portion. Referring to FIG. 11, the working portion is shown such that a rear staple 46 together, with the rest of the staples, may forcibly protrude towards the wall 44 of the artery. Upon the staple protruding, the staple pierces the wall 40 of the graft and the wall 44 of the artery and thus affixes the proximal end 20 of the graft at the proximal end 36 of the artery. An example of this situation can be seen in FIG. 12 staples of the rear group are not shown). Upon affixing both ends of the graft, the graft is secured within the artery and the barrel portion 16 may be evacuated from the artery 32 by retracting it in the proximal direction P as shown in FIG. 13.

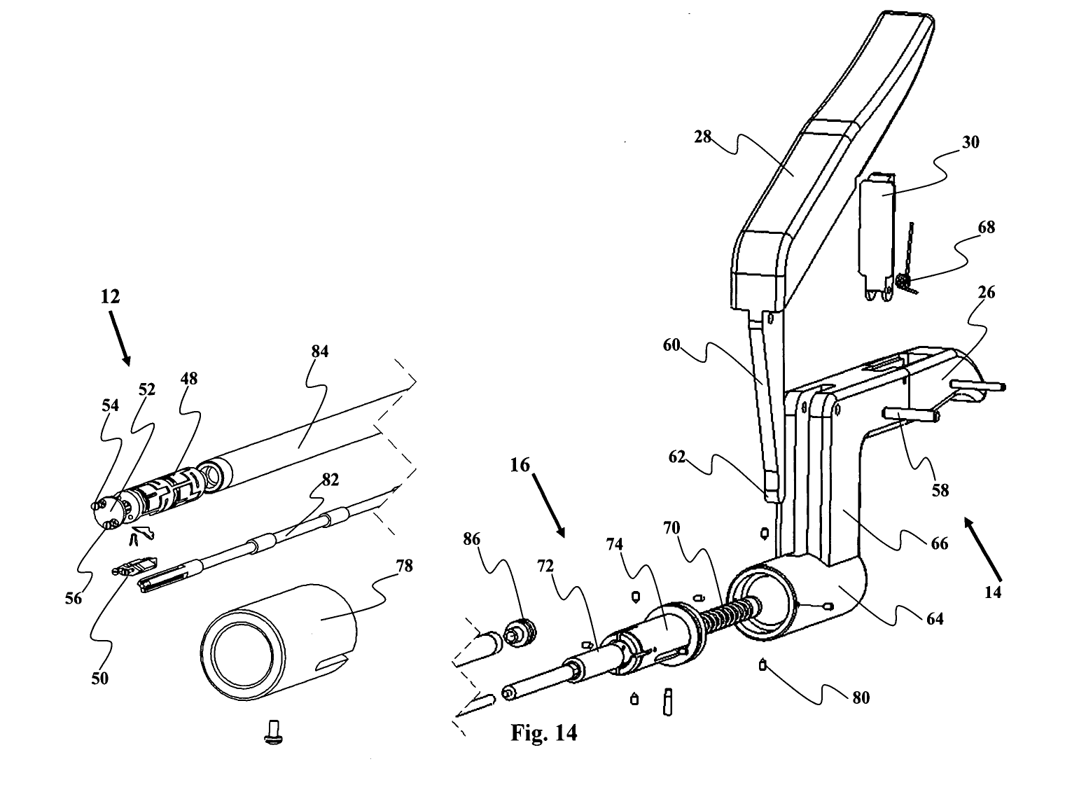

Referring to FIG. 14, an exploded view of the device according to some embodiments is shown. The device comprises the working portion 12, the handle portion 14, and the barrel portion 16. The working portion 12 comprises a main body portion 48 preferably adapted to accommodate therein a plurality of pivoting wings and pivoting levers, which may be divided into frontal groups and rear groups. The frontal and rear groups are respectively preferably intended to displace a plurality of the frontal staples and a plurality of the rear staples towards the vessel wall and thus to subsequently affix the distal and the proximal end of the graft to the vessel wall. FIG. 14 partially shows one such frontal group of wings and levers, designated by a common numeral 50. The rear group of wings and levers is not shown. The frontal end of the main body portion may be closed by a plug 52, which may be securable on the main body portion by fasteners (e.g., screws) 54, 56 or fastening means.

The handle portion may comprise lower handle 26 and upper handle 28. The handle 26 can be pivoted about an axle 58 upon manually pressing the handles together. The upper handle 28 may be provided with a lever arm 60 terminating in end 62. The lower handle 26 may be provided with a tubular housing 64 which may be connected to the handle 26 via an intermediate portion 66. When the handle portion is assembled, the lever arm 60 is preferably accommodated within the intermediate portion 66, with the possibility for displacement therein such that upon pressing the handles, the end 62 can reciprocate within the housing 64. The handle portion may be further provided with the swiveling latch 30, which is intended for preventing the handles from inadvertent encroachment. When the handle portion is assembled and the device is ready for operation, the latch may be forcibly held in a locked up-right position by a spring 68. To operate the device, the handles can be unlocked by moving the latch against the spring 68 from the up-right position to a horizontal position.

Some embodiments of the handle portion may further comprise a spring guide shaft 70, an inner guiding bushing 72, a collet member 74, a biasing spring 76, and an external guiding bushing 78. A plurality of fasteners (e.g., screws or other fastener types/means) 80 may be provided for securing the collet member and/or the other elements of the handle portion at the housing 64.

The barrel portion may comprise a pushing rod 82, a barrel 84, and an entry port 86.

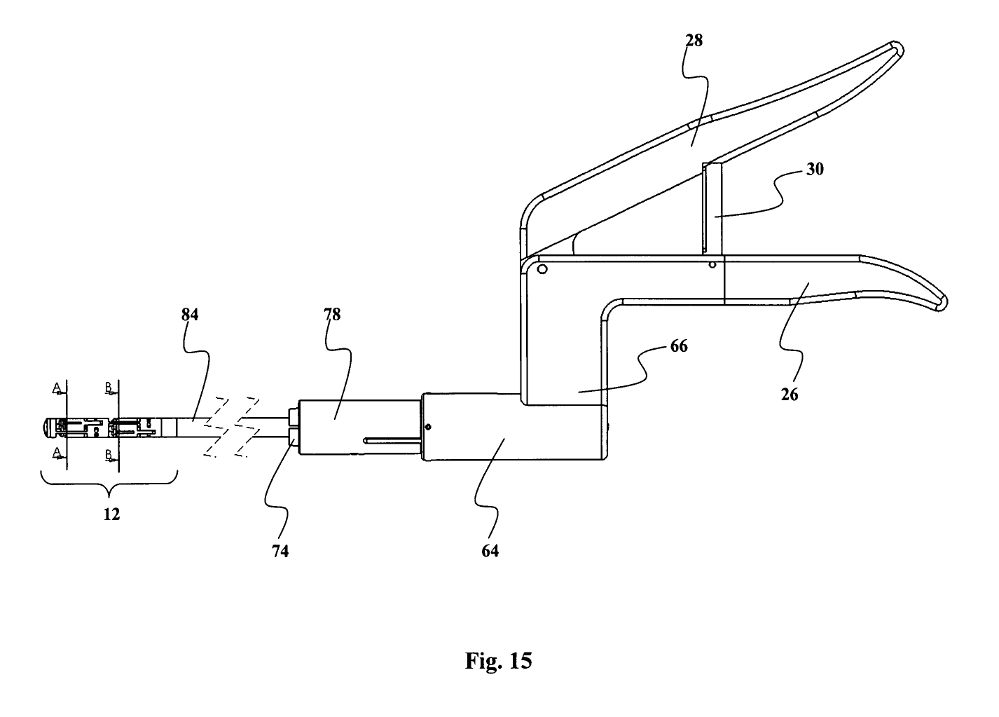

FIGS. 15, 16, 17 and 18 illustrate the construction of the working portion, according to some embodiments. FIG. 15 shows a side view of the device with two cross-sectional planes A-A and B-B drawn through the frontal and the rear groups of the wings and levers.

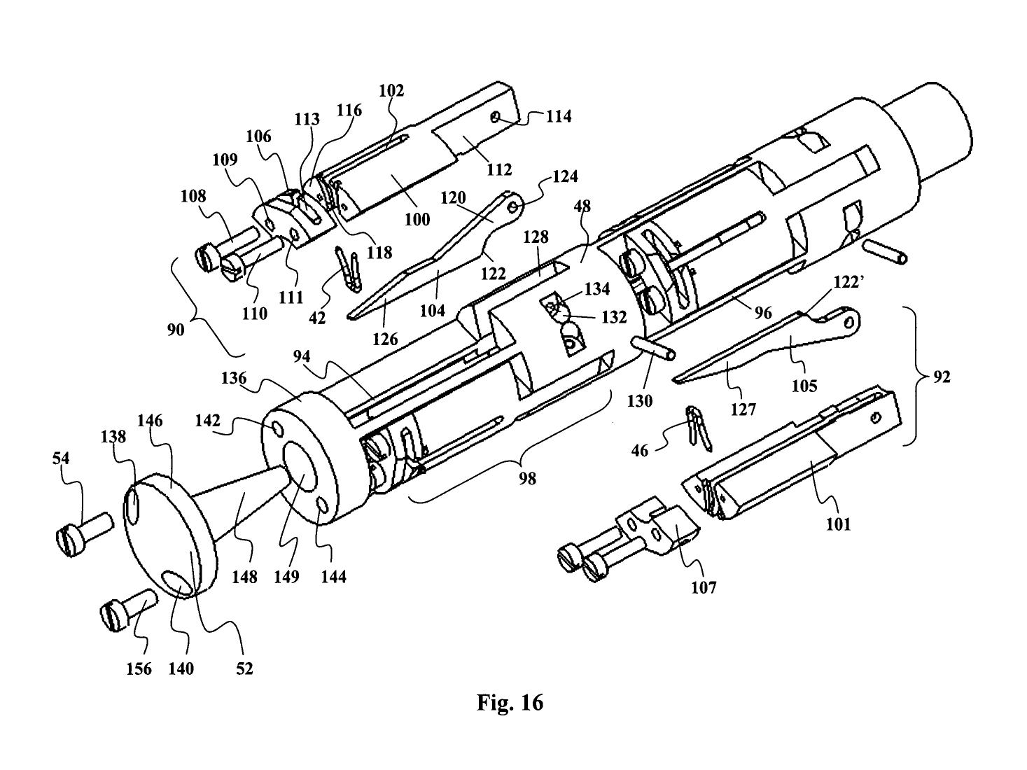

FIG. 16 depicts an exploded view of the working portion with its main elements, which may comprise a main body portion 48 accommodating therein, for example, the frontal and rear groups of wings and levers. The main body portion may include a tubular shape, which may include appropriate longitudinally directed cut outs for receiving at least one of and preferably both of the frontal and the rear groups of wings and levers. It will be apparent from the figure that a bore may be provided which extends through the main body and preferably receives therein the pushing rod and preferably provides for the pushing rod's linear and/or rotational displacement within the main body portion.

Altogether, the main body portion, according to some embodiments, can accommodate three frontal groups of wings and levers and three rear groups of wings and levers. In FIG. 16 an exploded view of one frontal group is shown, namely a frontal group 90 and one rear group, namely a rear group 92. Plug 52 with fasteners 54, 56 is also shown. As mentioned before, the main body portion is provided with longitudinal cut-outs, in which the frontal and the rear groups of the wings and levers reside with the possibility for pivoting. The frontal group 90 is shown to reside in one of the cut-outs 94. The rear group 92 is shown to reside in another cut-out 96 (for example). One should appreciate that the remaining frontal and rear groups of wings and levers may also reside within respective longitudinal cut outs, see for example the frontal group 98.

The frontal group 90 may include a pivoting wing 100 provided with a longitudinal slot 102, a pivoting lever 104, and a frontal slotted element 106, which may be securable at the wing by one or more fasteners (e.g., screws) 108, 110 (for example). The pivoting wing may include an elongated shape, which may terminate at one side thereof by a root end 112 provided with a hole 114 for receiving a pivot axle. The wing may terminate at the opposite side thereof by a butt end 116 having a cut out 118. This cut out may be configured and dimensioned such that a U-shaped staple 42 can be loaded into the cut out when the frontal group 90 is being assembled and deployed in the cut out 94. When the device is assembled, the pivoting lever 104 may be located in the slot 102, which allows pivoting of the lever. The frontal slotted element may include a couple of holes 109, 111; and in the butt end of the wing, there may be provided corresponding threaded bores. The staple preferably remains in place since, when the fasteners 108,110 are threaded, the frontal element 106 may close the staple and apply elastic force thereon due to a slot 113 made in the frontal slotted element 106. By virtue of this, the staple is pressed towards the butt end and remains substantially in place. At the same time, this elastic force, according to some embodiments, does not prevent the possibility for radial sliding of the staple along the cut out 118, and thus the staple can be forced to exit from the wing when the lever 104 pivots.

The pivoting lever 104 may include a rear end 120 which may be provided with a hole 124 and curved side 122. The pivoting lever may be provided with a front end 126, which preferably exerts a pushing force on the frontal staple 42 when the lever is urged to pivot. In the wing body, a longitudinal slot 128 may be provided such that the root end 112 of the wing 100 can be deployed in this slot, which allows for forcible pivoting of the wing. A pivot axle 130 is provided and a depression 132 is made in the main body portion. A hole 134 may be included in the depression such that when the wing 100 and the lever 104 are deployed in the main body portion, the holes 114, 124 and 134 align to allow insertion of the pivot axle 130.

Within the main body portion (according to some embodiments) are preferably deployed altogether three frontal groups of wings and levers and three rear groups of wings and levers. All these groups are preferably designed and assembled similarly to what has been discussed above with reference to the frontal group 90. The frontal groups are located in the frontal region of the main body portion and may be symmetrically distributed along its circumference with an interval of 120 degrees. An exploded view of the rear group 92 is shown in FIG. 16. It should be appreciated that the group 92 may be arranged similarly to the frontal group 90. For the sake of brevity, only some of elements of the rear group 92 are described further. It is seen, for example, that 92 includes rear staple 46, a rear pivoting wing 101, and a rear pivoting lever 105 having a front end 127 and a curved side 122′ and a slotted element 107. Three such rear groups may be provided and they are deployed in the rear region of the main body portion behind the frontal groups and are remote therefrom. Similarly to the frontal groups, the rear groups may also be symmetrically distributed along the circumference of the main body portion with an interval of about 120 degrees (for example), however they are preferably located in such a manner that each rear group is displaced at about 60 degrees (for example) with respect to each frontal group such that, in some embodiments, none of the rear groups is aligned with any of the frontal groups. Thus, it is possible to independently actuate the frontal groups and the rear groups according to some embodiments of the invention.

During assembly of the working portion, the plug 52 may be secured at a forward end 136 of the main body portion by fasteners 54, 56, which pass through corresponding holes 138, 140 and are threaded into corresponding threaded bores 142, 144 made in the forward end. The plug has a cover portion 146, in which the holes 138, 140 may be provided and may include a cone stem portion 148, which protrudes into the main body portion via a window 149 in the forward end. The direction of the cone portion, as well as its length, may be selected in such a manner that when the fasteners 54, 56 have been fastened in the main body portion, the cone portion pushes the front ends of the levers of the frontal groups and urges the levers to pivot in a clockwise direction (for example). This in turn urges the staples loaded in the frontal wings to slightly protrude out of the working portion. It will be appreciated that upon assembling the working portion and connecting it with the intermediate barrel portion, a graft may be placed on the barrel portion such that it also covers the working portion. Therefore, fastening of fasteners 54, 56 and pivoting of the frontal levers may be associated with the protrusion of frontal staples from the working portion into the graft. This preferably provides provisional connection of the graft to the working portion as mentioned above with reference to FIG. 2.

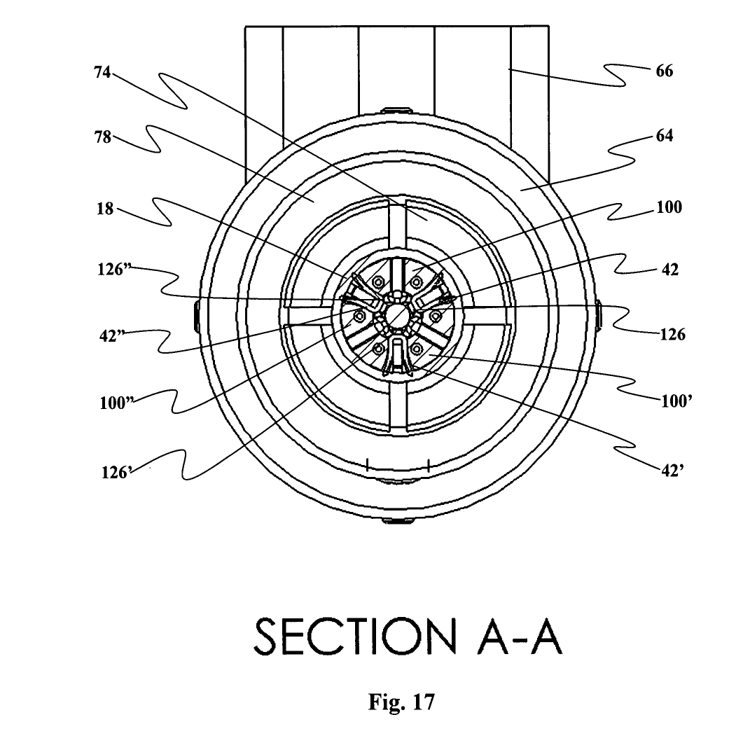

Referring to FIGS. 17-20, the above arrangement of the frontal and the rear groups of wings and levers as well as provisional connection of the graft to the device will be discussed in more detail.

FIG. 17 is a cross-sectional view taken along plane A-A shown in FIG. 15. In FIG. 17 three groups of frontal wings 100,100′,100″ are shown as well as a plurality of frontal staples 42,42′,42″ and front ends 126,126′,126″ of the respective frontal levers. The above elements may be symmetrically distributed on the circumference of the main body portion with an interval of about 120 degrees (for example). The frontal staples preferably slightly protrude from the working portion into graft 18, thus providing provisional connection between the distal end of the graft and the working portion. The rest of the previously described elements of the handle portion, such as housing 64, intermediate portion 66, collet member 74, and external guiding bushing 78, are shown as well.

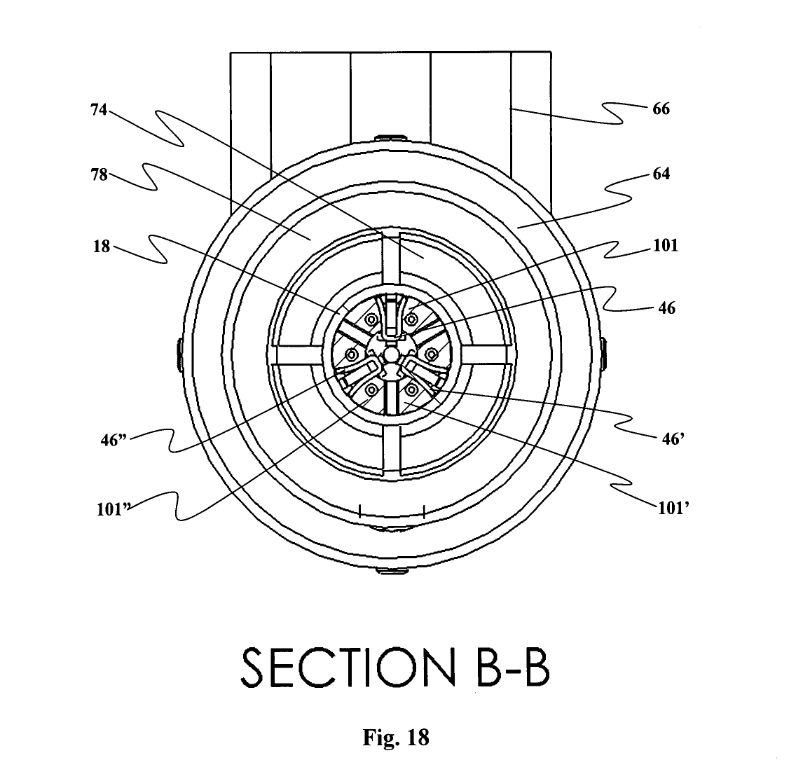

FIG. 18 is a cross-sectional view taken along plane B-B shown in FIG. 15. In FIG. 18, three groups of rear wings 101,101′, 101″ are shown as well as a plurality of rear staples 46,46′,46″ and front ends 127,127′,127″ of the respective rear levers. The above elements of frontal groups may be symmetrically distributed along the circumference of the main body portion with an interval of about 120 degrees (for example). However, the location of each rear group is preferably different from the frontal group, i.e., each rear group (for example) is preferably displaced at about 60 degrees (for example) with respect to each frontal group, such that none of the rear groups is in alignment with any of the frontal groups. In such an embodiment, none of the rear staples protrudes into graft 18 (as shown).

The rest of the already mentioned elements of the handle portion, such as housing 64, intermediate portion 66, collet member 74, and external guiding bushing 78, are shown as well.

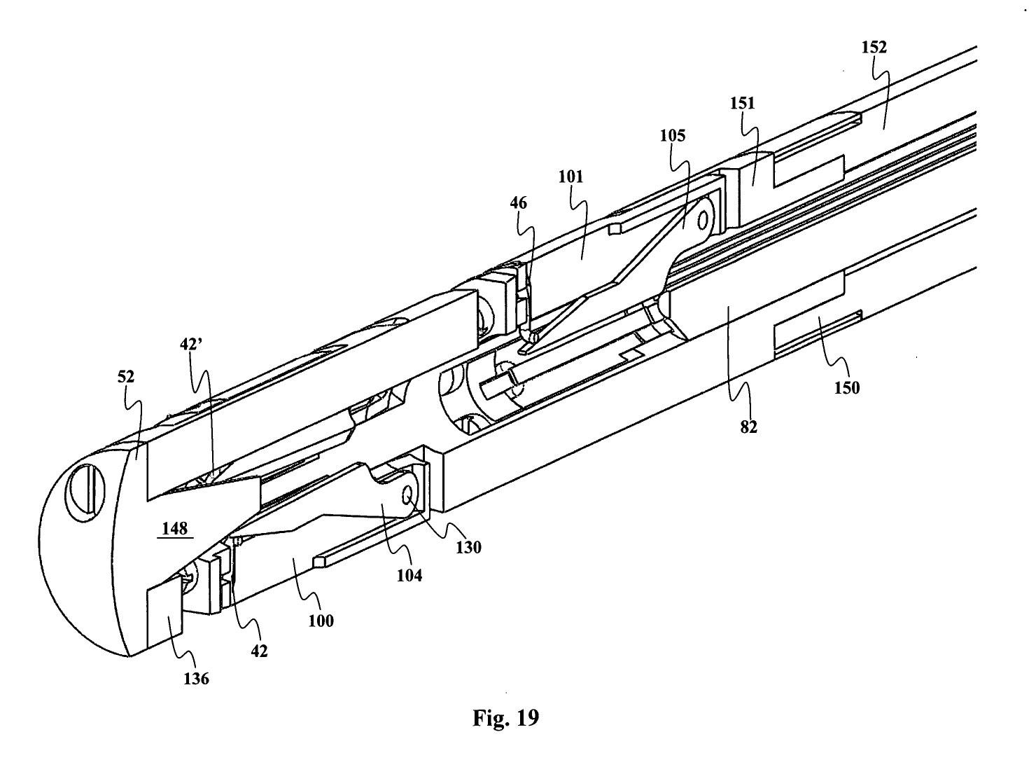

FIG. 19 shows that securing the plug 52 on the forward end 136 of the main body portion, in some embodiments, causes the cone portion 148 to approach front ends of the frontal levers and to urge them to pivot about their respective pivot axles. The levers pivot in respective slots made in the respective wings. The pivoting motion of the levers force the frontal staples to slightly protrude out of the main body portion such that sharpened ends of the staples pierce the wall of the graft 18. In FIG. 19 frontal staple 42, 42′, wings 100, lever 104 are also shown.

FIG. 19 also shows some elements of the rear groups, such as rear staple 46, one rear wing 101, and rear lever 105.

A rearmost portion 151 of the main body portion may be configured with a circular slot 150, which receives a forward most end 152 of the barrel. The slot 150 and the end 152 may be threaded such that the barrel can be detachably connected to the main body portion. It should be appreciated that upon the graft 18 being placed on the barrel, it may cover the working portion as well as the barrel. Furthermore the pushing rod 82 is shown extended through the working portion.

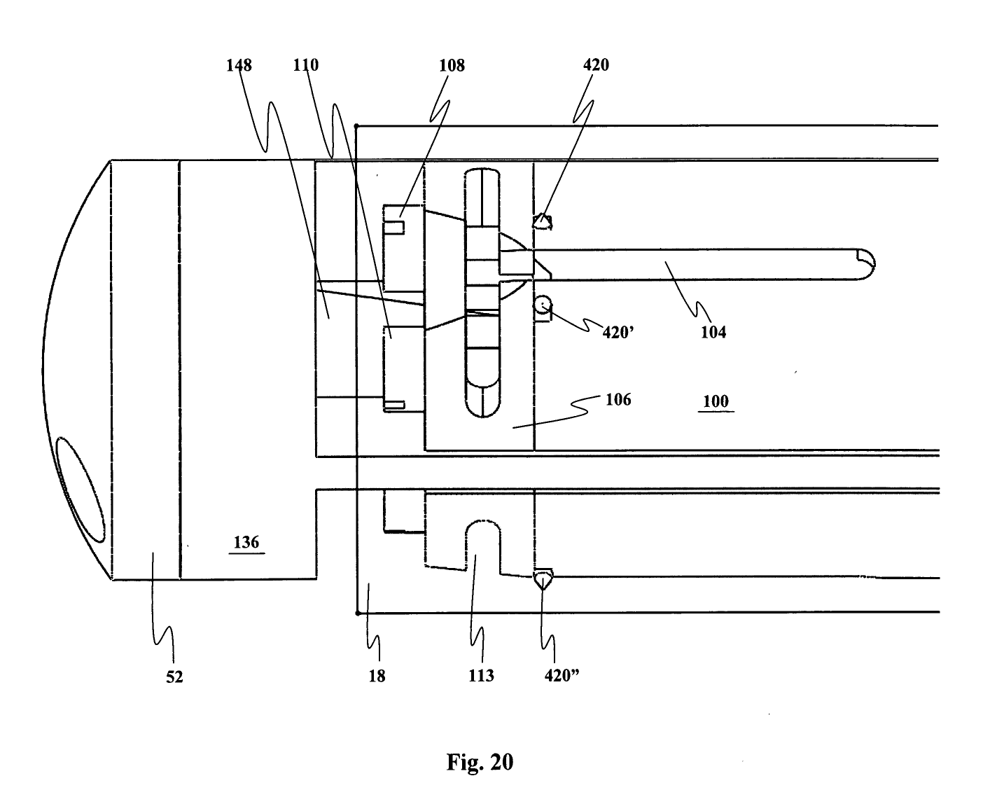

FIG. 20 depicts how sharpened ends 420, 420′, 420″ of the frontal staples slightly protrude into a wall of the graft 18 that is put over the working portion, thus providing the provisional connection between the graft and the working portion, according to some embodiments. It also can be appreciated that the staples may be retained between butt ends of the respective wings and slotted frontal elements. One may also appreciate that similar combinations of elements exist in all frontal groups of pivoting wings and levers.

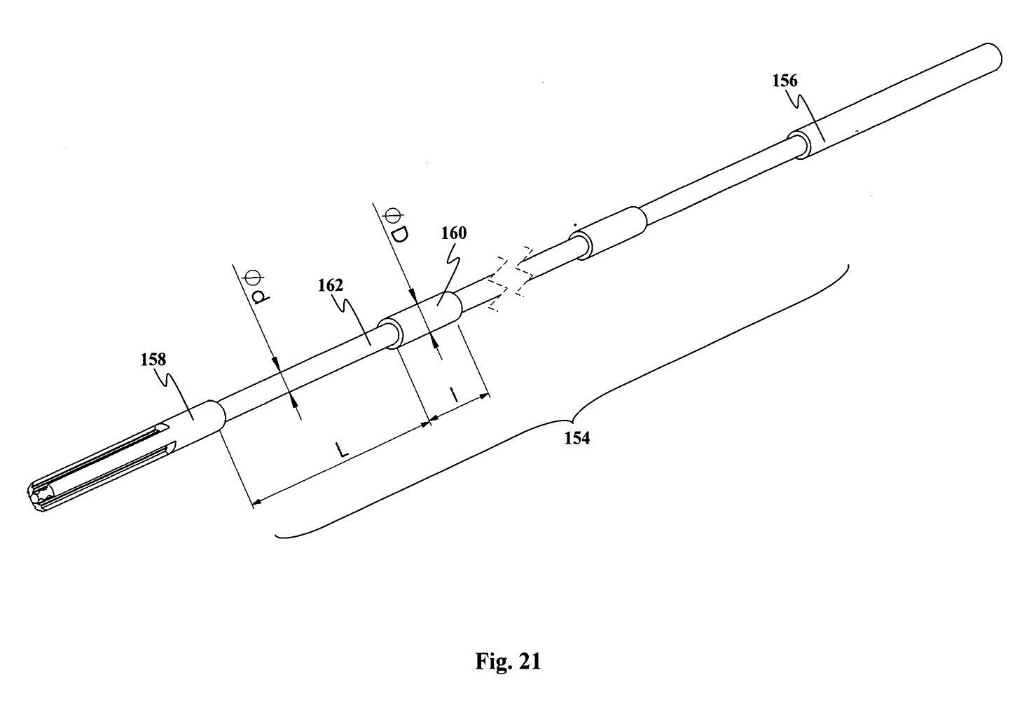

Referring to FIGS. 21-25, the actuating of the frontal or rear groups of wings and levers by the displacement of the pushing rod along the working portion will be described. Pivoting of the levers, according to some embodiments, causes protrusion of the staples in a radial direction towards the graft wall and preferably results in affixing the graft to the vessel.

In FIG. 21, the pushing rod 82 is depicted and may comprise a shaft portion 154, a rear end 156 and a forward head 158. The shaft portion may be long and relatively thin; and in order to render it sufficiently rigid, it is divided into a plurality of regions 160 of a larger diameter D, preferably interspersed with a plurality of regions 162 of a smaller diameter d. For example, the length of the regions 160 may be l and the length of the regions 162 may be L. In practice, the length l can be equal with the length L. The pushing rod can be manufactured from metallic or plastic material, e.g. from stainless steel 316L or from Polycarbonate.

In some embodiments, the length of the shaft portion may be about 530 mm, the length l of the regions 160 and the length L of the regions 162 may be about 30 mm, the diameter D may be about 4-5 mm and the diameter d may be about 1.5-2 mm.

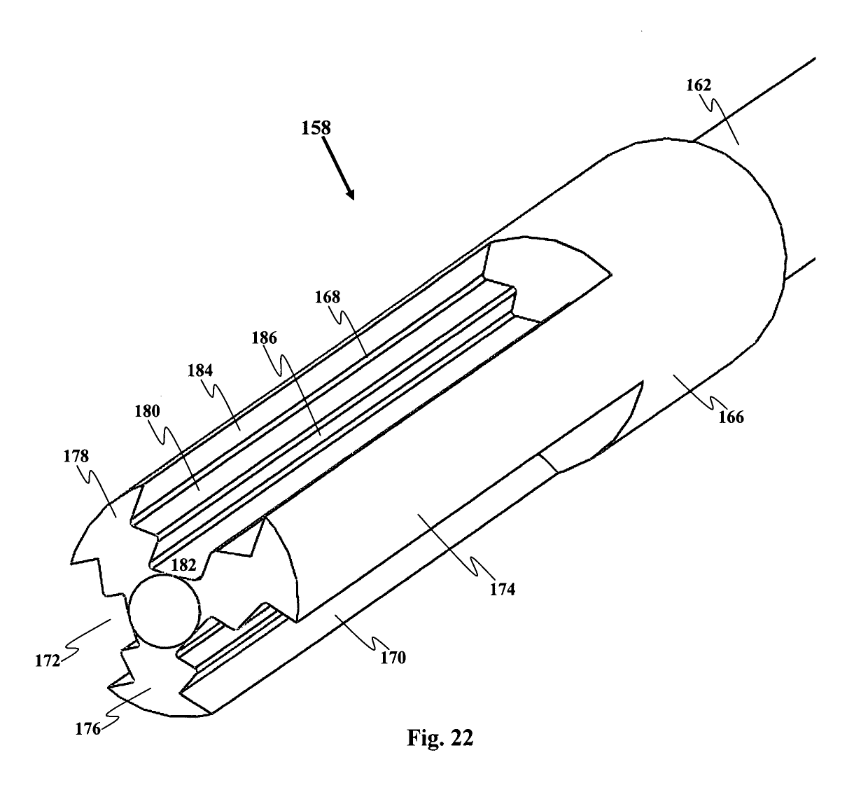

With reference to FIG. 22, construction of the forward head 158 will be discussed. The forward head is situated adjacent to the region 162, and may comprise a cylindrical rear portion 166 having diameter D, as the regions 160 of the shaft portion. The reminder of the head portion may also have diameter D; and in the bulk thereof, three symmetrically arranged longitudinal recess zones 168, 170, 172 may be included. The recess zones are preferably delimited by three respective non-recessed zones 174, 176 and 178, such that recess zone 168 may be delimited by adjacent non-recessed zones 174 and 178; recess zone 170 may be delimited by adjacent non-recessed zones 174, 176; and recess zone 172 may be delimited by adjacent non-recessed zones 176, 178. The recessed zones and the non-recessed zones are preferably arranged symmetrically on the circumference of the forward head such that they are spaced from each other by about 60 degrees (for example). Each recess zone may be defined by a ledge 180 and a ledge 182, which may be arranged as steps having vertical walls 184, 186. The recess zones and the non-recessed zones are preferably configured in such a manner that when the pushing rod is forcibly advanced along the working portion, the recess zones are situated (preferably at all times) between either the frontal groups or the rear groups of pivoting wings and levers; thus, preferably with no engagement between the recess zones and the pivoting elements. At the same time, the outside diameter D of the working head may be selected in such a manner that the circumference of the non-recessed zones may engage the wings and levers either of the frontal groups or of the rear groups and therefore, may actuate them and urge the wings and levers to pivot (for example).

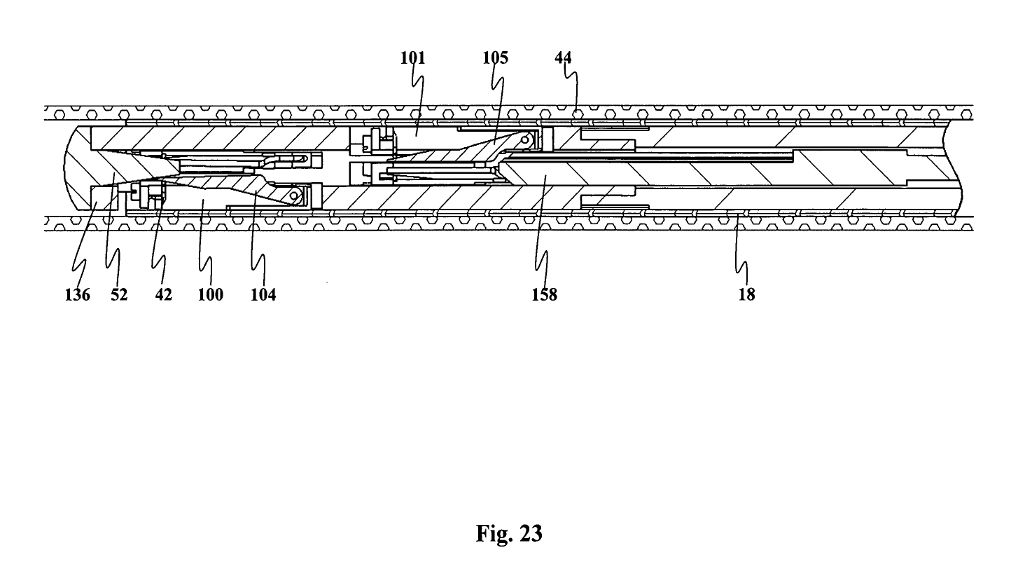

FIGS. 23-28 show the working portion 12 after it has been brought into a vessel, e.g., in artery 44. The graft 18 is put on the barrel 84 and covers the working portion (for example).

The forward most end 152 of the barrel 84 may be threaded into the rear most portion 151 of the main body portion, which receives the forward head 158 of the pushing road.

FIG. 23 shows a situation when the graft is brought into the vessel with the aim of affixing the distal end of the graft to a position in the vessel. This situation has been depicted already with reference to FIG. 6.

The forward head of the pushing rod is at an initial position, in which it neither engages the rear groups nor the frontal groups of the wings and levers (for example). Wing 100 and lever 104 of the frontal group, as well as wing 101 and lever 105 of the rear group, are seen as being received (preferably fully) within the respective cut-outs of the main body portion and are not pivoted yet. Frontal staple 42 protrudes slightly into graft 18, for example, after plug 52 has been secured at the forward end 136 of the main body portion to provide provisional connection between the graft and the working portion. In this embodiment, the graft is not yet affixed to the vessel. The rear staples, in such an embodiment, do not protrude into the graft.

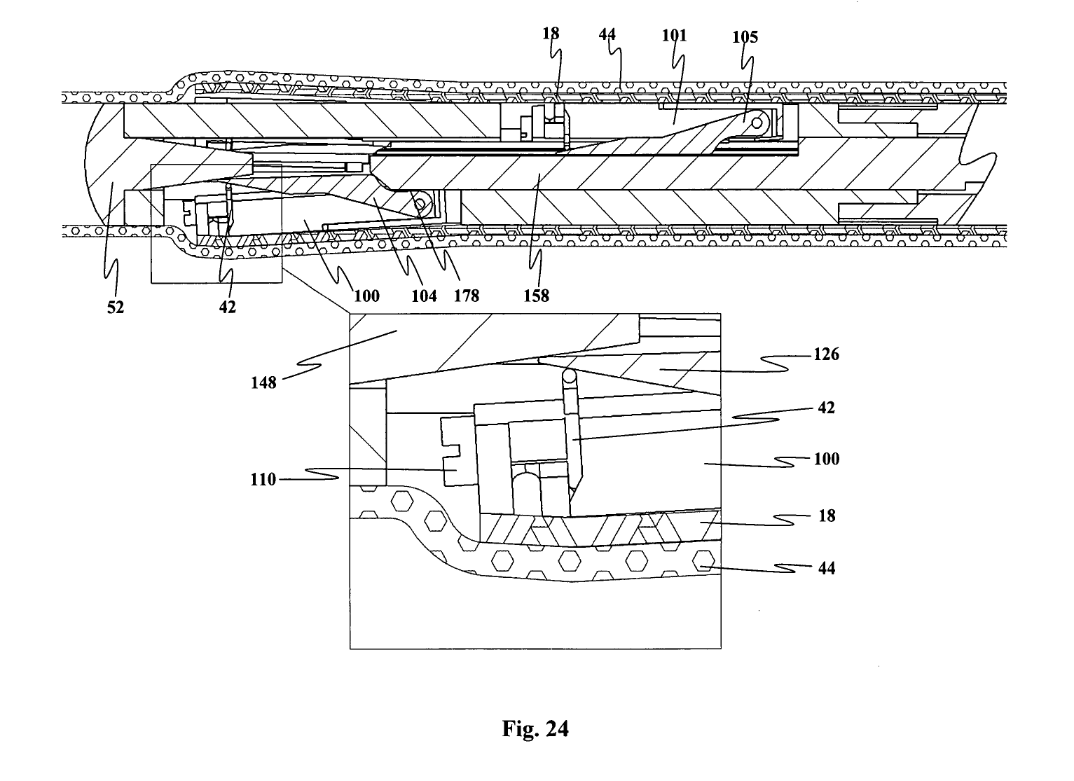

FIG. 24 shows a situation in which the pushing rod has been further advanced distally by the handle portion. The advancement mechanism is described below. The pushing rod may be angularly oriented within the barrel in such a manner that when the pushing rod is displaced distally, the circumference of the non-recessed zones of the forward head 158 engages the wings of the frontal groups and causes them to pivot clockwise (for example). FIG. 24 shows how the wing 100 has been pivoted outside of the working portion by the circumference of the non-recessed zone 178. Also shown, the pivoted wing has stretched the graft in radial direction (for example). During pivoting, the wing 100 preferably does not displace the frontal staple 42. The lever 104 has not yet been pivoted, according to the present embodiment, and remains in the same position within the wing as shown in FIG. 23.

Not shown in FIG. 24, but what will be appreciated by one of skilled in the art, is that all three wings of each frontal group have been engaged and actuated simultaneously and in the same manner as the wing 100, according to some embodiments.

As shown, and according to some embodiments, the rear groups of the pivoting wings were not actuated since they are not aligned with the frontal groups. They are arranged within the working portion in such a manner that, during advancement of the pushing rod, the recessed zones of the forward head preferably freely pass along the rear groups without engaging them, thus not actuating the wings and the levers. Pivoting wing 101 and pivoting lever 105 are shown located within the working head and not actuated yet. It will be appreciated, although not shown, that all three rear groups of wings and levers have not been actuated yet and remain so far in place.

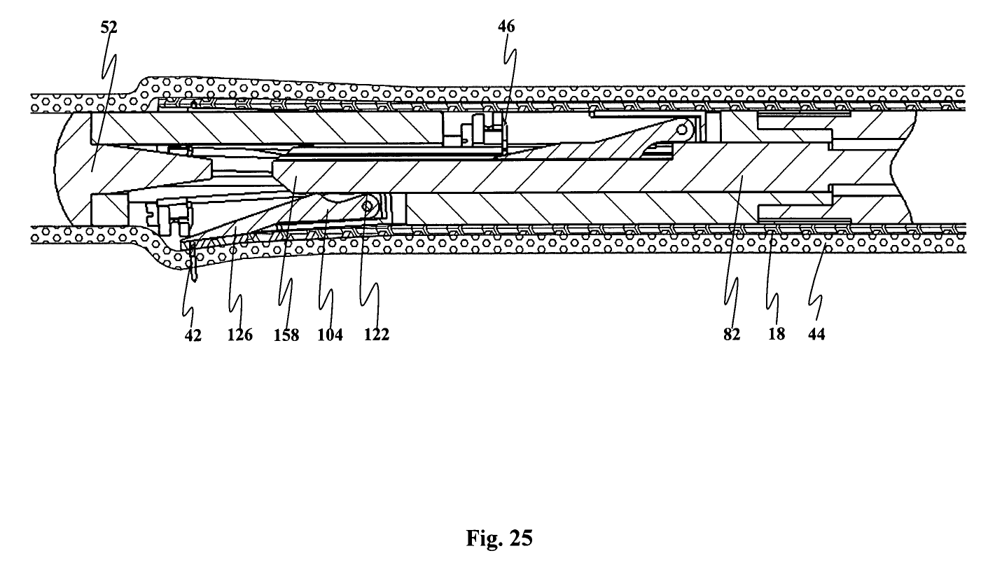

FIG. 25 shows a third position of the pushing rod 82, according to some embodiments, in which it has been advanced distally still further, such that its forward head approached curved side 122 of the lever 104 and caused it to pivot. Due to the pivoting, the front end 126 of the lever 104 forces the frontal staple 42 to displace from the cut-out provided in the butt end of the wing. The staple may be displaced towards the graft wall to an extent that, at the end of the lever pivoting motion, it pierces the vessel wall. By virtue of this provision, the distal end of the graft becomes affixed to the vessel. It is not shown in FIG. 25, but is shown in FIG. 16, that the cut-out 118 made in the butt end 116 of the wing 100 may be configured with a flaring exit. This configuration, which is better seen in FIGS. 17, 18, causes the sharpened ends of the staple to slightly bend outside when the staple exits from the working portion. The bent ends eventually render affixing of the graft to the vessel more reliable. Furthermore, the protrusion of the staple ends within the graft may be facilitated in that the graft remains stretched in a radial direction by the wing when the staple pierces it. It will also be appreciated that all groups of the wings and levers can be designed with flaring exit.

It will also be appreciated that all three frontal groups of the wings and levers may be engaged and actuated simultaneously, and they are forced to pivot in a similar way and with the same result (according to some embodiments).

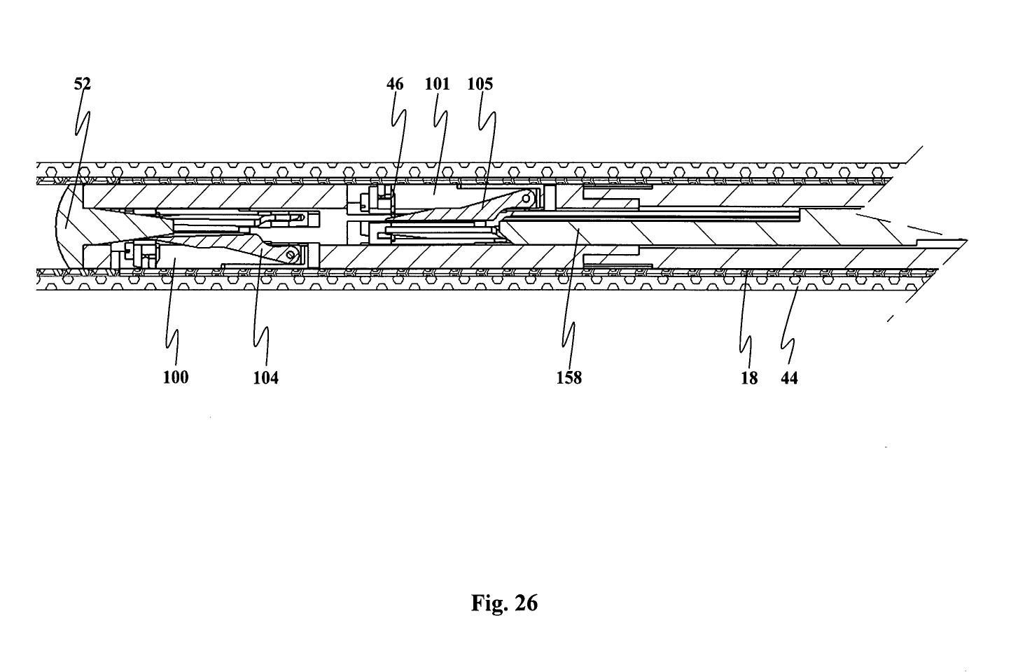

FIG. 26 shows a situation when the barrel portion and the working portion are brought to the proximal end of the vessel and the device is ready for affixing the proximal end of the graft to the proximal end of the vessel according to some embodiments. After the distal end of the graft has been affixed, the proximal end of the graft preferably disconnects from the handle portion. The barrel portion is then free for retracting proximally along the graft until the working portion is brought to a new location, which is the proximal end of the graft to be affixed to the proximal end of the vessel. Furthermore, before affixing the proximal end of the graft, the pushing rod may be forcibly rotated within the barrel portion to an extent that, upon distal protracting of the barrel portion, the non-recessed zones engage and actuate the rear groups of the wings and levers.

FIG. 26 shows that the pushing rod protracted distally in a position in which it is close to the wing 101. Upon distal displacement, first the wing 101 and then the lever 105 may be engaged and actuated, which, in turn, may result in protruding the rear staple 46 into graft 18 and then into vessel wall 44.

FIG. 26 also shows that upon bringing the working portion to the new location, the wing 100 and lever 104 of the frontal groups may return to the non-pivoted position and may be fully received in the main body portion. It should be appreciated that all three frontal groups have returned to the same non-pivoted position.

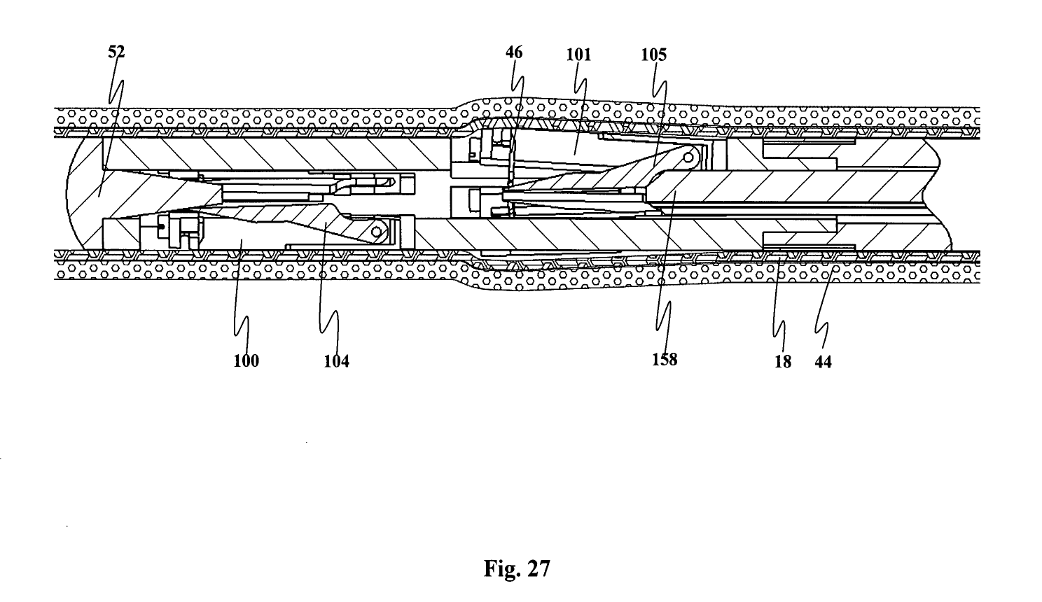

FIG. 27 shows the pushing rod after it has been further protracted, in which the forward head 158 engages the wing 101 to cause its pivoting outside of the working portion, according to some embodiments. The pivoting motion is associated with stretching the graft 18 in a radial direction as explained above in connection with FIG. 25. The lever 105 may not yet be actuated and the rear staple 46 remains in place. It will be appreciated that despite describing only elements of one rear group, all three rear groups have been engaged and actuated simultaneously and in a similar way.

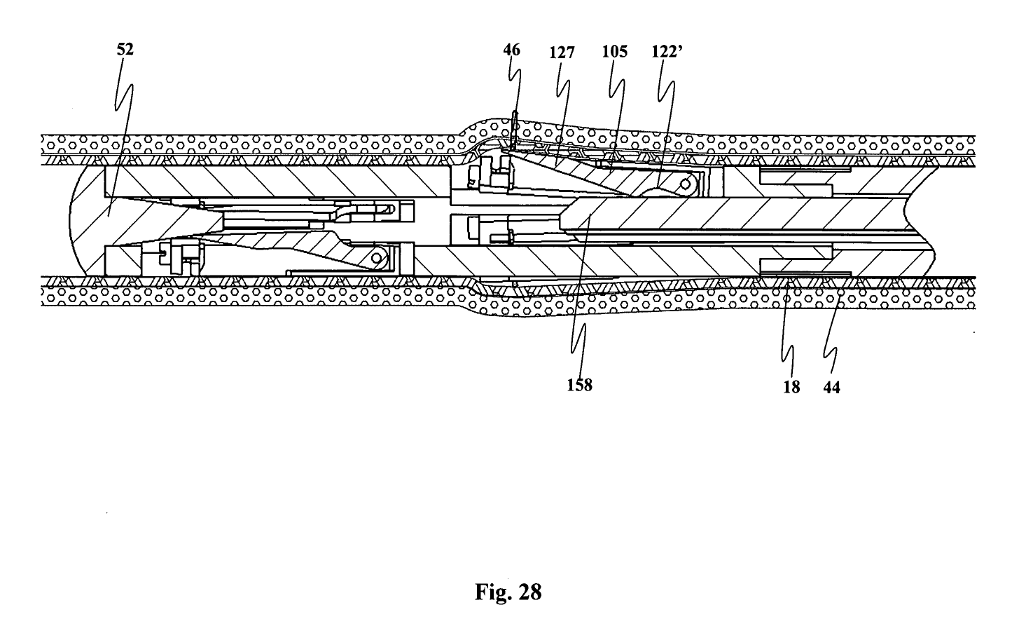

FIG. 28 shows a still further advanced position of the pushing rod, in which its forward head 158 contacts the curved side 122′ of the lever 105, according to some embodiments. This urges the lever to pivot with respect to wing such that forward end 127 of the lever pushes the rear staple 46 to protrude into graft 18, to pierce the vessel 44 and eventually to affix the proximal end of the graft to the proximal end of the vessel. It will be appreciated that despite describing only elements of one rear group, all three rear groups have been engaged and actuated simultaneously and in a similar way.

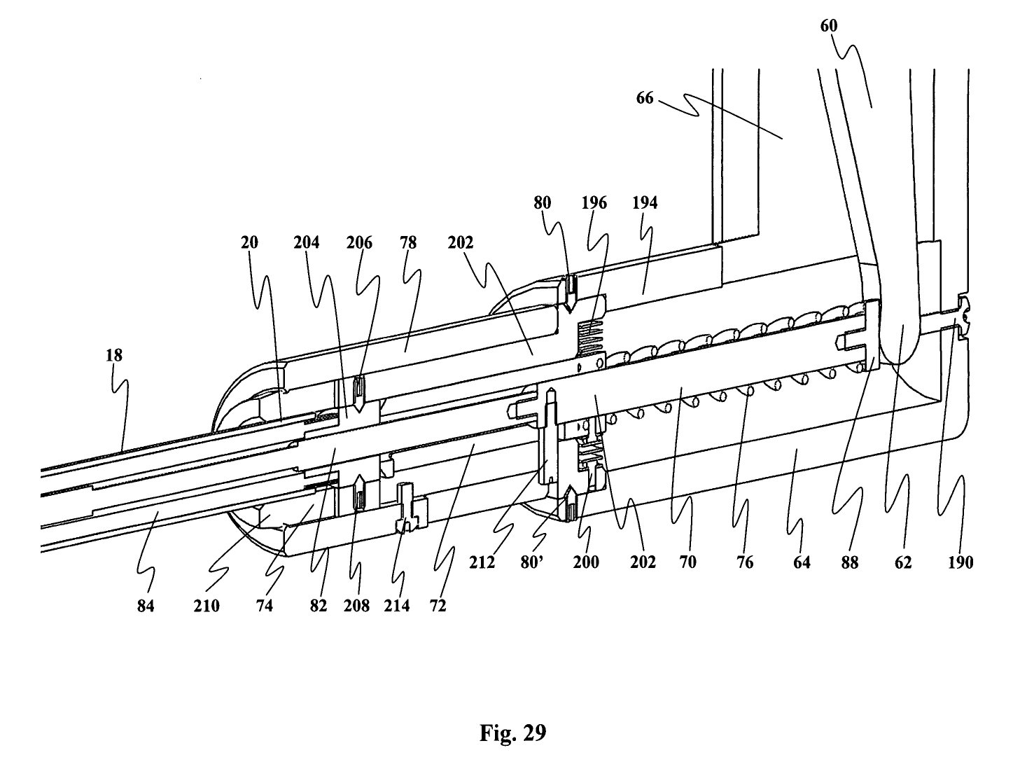

FIG. 29 shows the handle portion when it is assembled and the graft when it is placed over the barrel. The rear end of the graft may be provisionally connected to the handle portion. It is seen that the handle portion may comprise housing 64 with the intermediate portion 66 receiving therein the lever arm 60. The lever arm can pivot within the intermediate portion upon pressing or release of handles 26, 28. Within the housing is located spring guide shaft 70 with the biasing spring 76 put thereon. An abutment disc 188 may be located between the proximal end of the spring and the upper end 62 of the lever arm. The arrangement is such that when the handles are brought closer together, the upper end 62 of the lever arm may advance distally within the housing against the spring; and when the handles are released, the upper end of the lever arm may be forced by the spring to retract proximally. The disc may be connected to the disc guide shaft 70, and thus relative rotational displacement is allowed between the spring guide shaft and the upper end of the lever arm. A limit screw 190 (for example) may be provided, which, upon threading into a rear wall 192 of the housing, protrudes towards the end 62 of the lever arm. The extent of retraction of the lever arm can be adjusted by the limit screw. Within the housing and adjacent to a forward end 194 thereof, a torsion spring 196 may be provided, which one end is connected by a fastener (e.g., screw) to the forward end 194 of the housing and which second end is connected by a fastener (e.g., screw) 200 to a rear end 202 of the guide shaft 70. The rear end of the inner guiding bushing 72 may be connected to the spring guide shaft 70 such that release of the torsion spring is associated with forcible clockwise rotation of the inner guiding bushing 72 together with the spring guide shaft 70. The inner guiding bushing and the spring guide shaft are rotated by the torsion spring relative to the other elements of the handle portion, i.e., relative to the collet member and to the external guiding bushing.

Secured at the forward end 194 by fasteners (e.g., screws) 80, 80′ may be a rear end of the collet member 74. The rear end of the pushing rod 82 may be connected with the forward end of the spring guide shaft 70 such that either reciprocating longitudinal displacement or rotational displacement of the guide shaft 70 is transferred to the pushing rod 82.

FIG. 29 shows that the handle portion, which may comprise the inner guiding bushing 72, the collet member 74 and the external guiding bushing 78, which may surround the pushing rod. A forward bushing 204 may be provided, through which the pushing rod passes. The forward bushing is preferably secured within the collet member by one or more fasteners (e.g., screws) 206, 208. The forward bushing may function as a slide bearing in which the pushing rod can longitudinally reciprocate and rotate. It is seen also that the rearmost end of the barrel 84 may be secured within the forward bushing 204 and that the graft 18 may be placed on the barrel. The proximal end 20 of the graft may be fastened between the barrel and a forward most end 210 of the collet member 74. Therefore, the graft may be provisionally connected to the handle portion. It will be apparent further that the graft disconnects from the handle portion when the handles are brought closer together, causing the external guiding bushing to be protracted distally with respect to the collet member. The handle portion may be fitted with a guiding screw 212 (for example), which may include a lower portion protruding within a longitudinal slot made in the collet member. Due to the guiding screw, it is preferably ensured that the external guiding bushing linearly displaces along the collet member. The handle portion may be fitted with a pushing pin 214, secured within a depression made in the forward most end of the spring guide shaft 70.

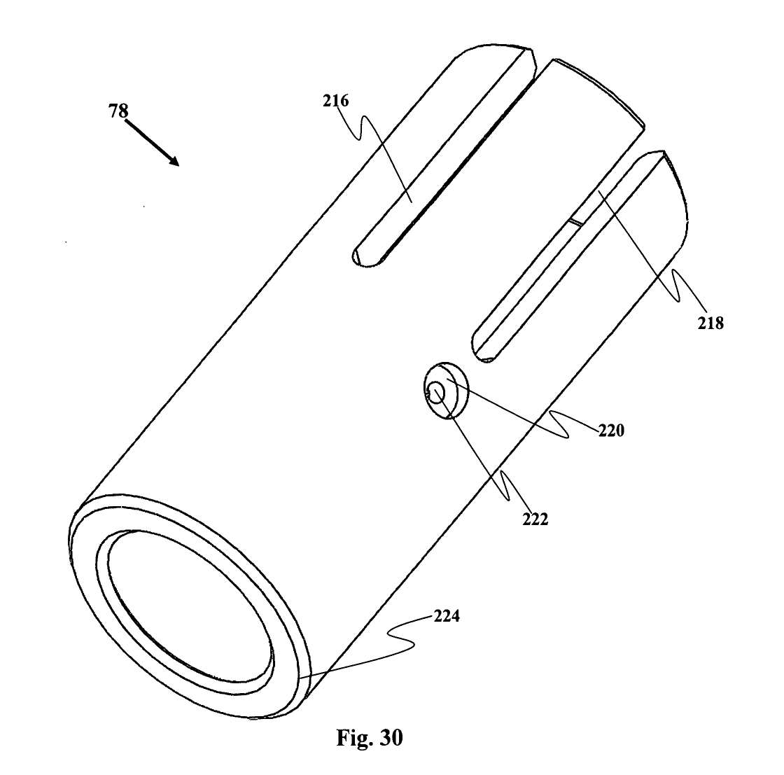

FIG. 30 shows the external guiding bushing 78. The external bushing may be configured as a tube, in which one end may be opened and which opposite end may be provided with two separate, longitudinal slots 216, 218. The slots are preferably parallel to the longitudinal axis of the external guiding bushing and may be separated at the circumference of the bushing by 60 degrees.

The width dimension of the slots may be selected to slightly exceed the outside diameter of the guiding pin 212 such that the pin can easily slide along the slots. Situated in front of one of the slots, a depression 220 may be provided, which terminates by a threaded bore 222 made in the wall of the bushing. The depression may be configured and dimensioned so as to fully receive the pushing pin 214. The opened end of the external guiding bushing may be configured with a lip portion 224. The lip portion may abut the forward-most end of the collet member such that the rear end of the graft is clamped between the collet member and the barrel.

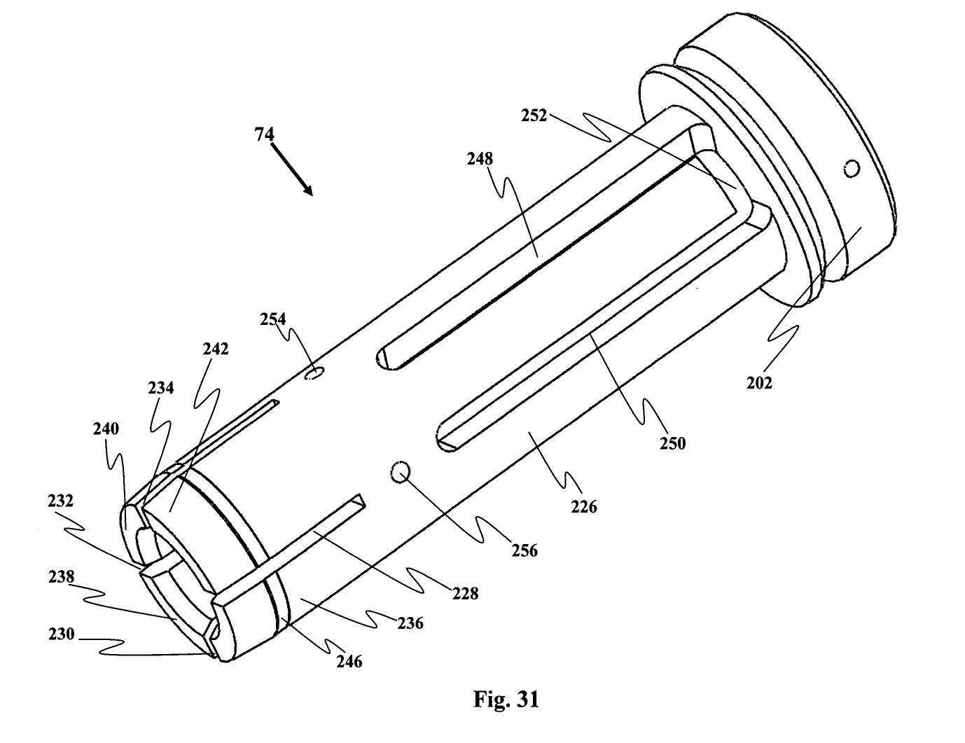

FIG. 31 shows the collet member 74. The collet member may comprise a rear end 202, which may be securable at the housing 64 of the handle portion. The collet member may include a stem portion 226, which may be configured as a tube in which a forward end may be provided with a plurality of longitudinal cuts 228-234 defining respective leaves 236-242. The inside diameter of the stem portion as well as the length and width dimension of the cuts may be selected in such a manner that when the handle portion is assembled and the external guiding bushing is put over the collet member, the leaves may be forcibly bent inwardly and clamp the rear end of the graft between the collet member and the barrel. In order to achieve reliable clamping, the stem portion is preferably provided with an annular groove 246, into which the lip portion 224 of the bushing 78 snaps. Two parallel longitudinal slots 248, 250 may be cut in the rear part of the stem portion. The slots are preferably separated at the circumference of the stem portion by 60 degrees. The longitudinal slots preferably communicate adjacent to the rear end 202 of the collet member via a transversal slot 252. The width dimension of the longitudinal slots and of the transversal slot may be selected to slightly exceed the outside diameter of the guiding pin 212 such that it can easily slide along all the slots.

Situated between slots and cuts, two holes 254, 256 are shown. The holes are intended for fasteners 206, 208.

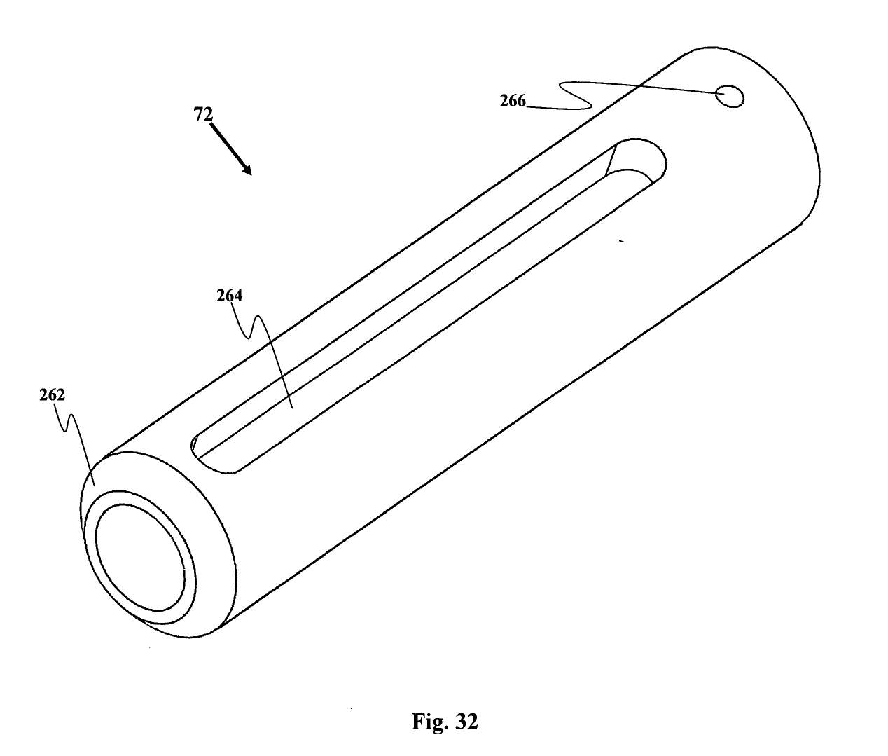

With reference to FIG. 32, the inner guiding bushing may be configured as a tube which one end 262 is faceted. A longitudinal slot 264 may be cut within the wall of the inner guiding bushing. The width dimension of the slot may be selected to slightly exceed the outside diameter of the guiding pin 212 such that it can easily slide along the slot. A hole 266 is shown in the rear part of the inner bushing member. This hole may be used to receive the fastener which connects one end of the torsion spring to the inner bushing member.

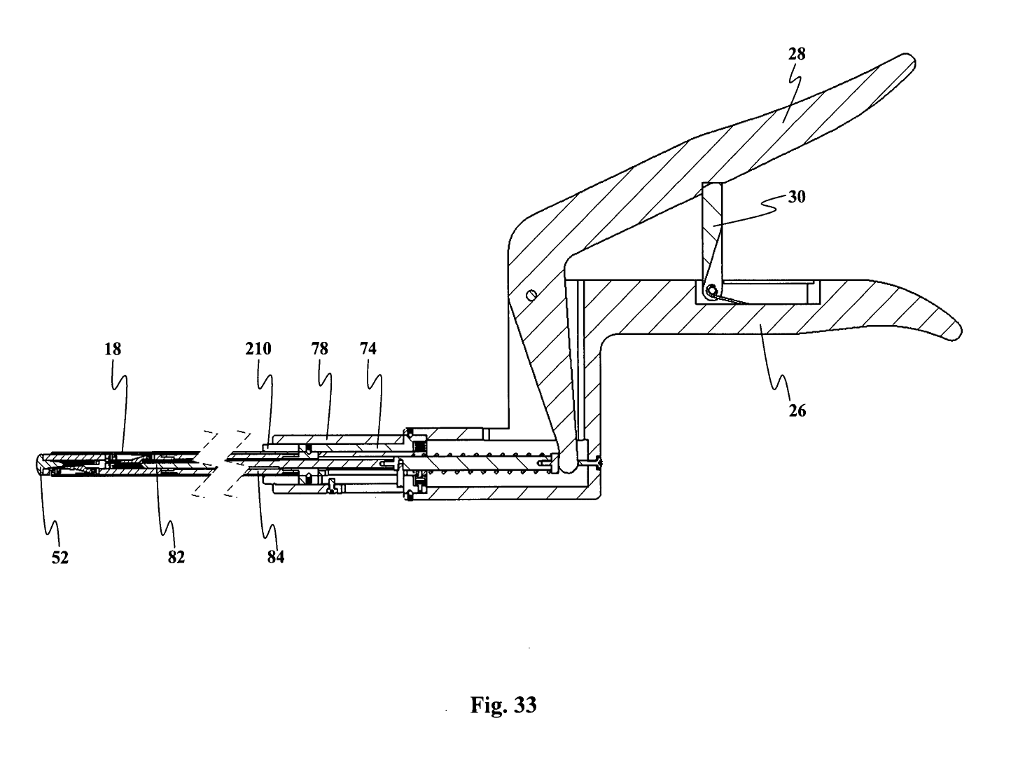

FIGS. 33-39 show how some embodiments of the device may be used in practice (methods of use according to some embodiments of the invention). In FIG. 33, the device is shown ready for operation. The handles 26 and 28 may be locked by the latch 30. The graft 18 is put on the barrel portion and the working portion is assembled such that plug 52 causes the distal end 22 of the graft to be provisionally connected to the working portion by a plurality of the frontal staples. At the same time, the handle portion may be assembled such that the lip portion of the external guiding bushing 78 presses on the forward-most end 210 of the collet member 74 and causes the proximal end of the graft to be provisionally fastened between the barrel 84 and the collet member. Pushing rod 82 is preferably not yet protracted towards the frontal groups of the wings and levers.

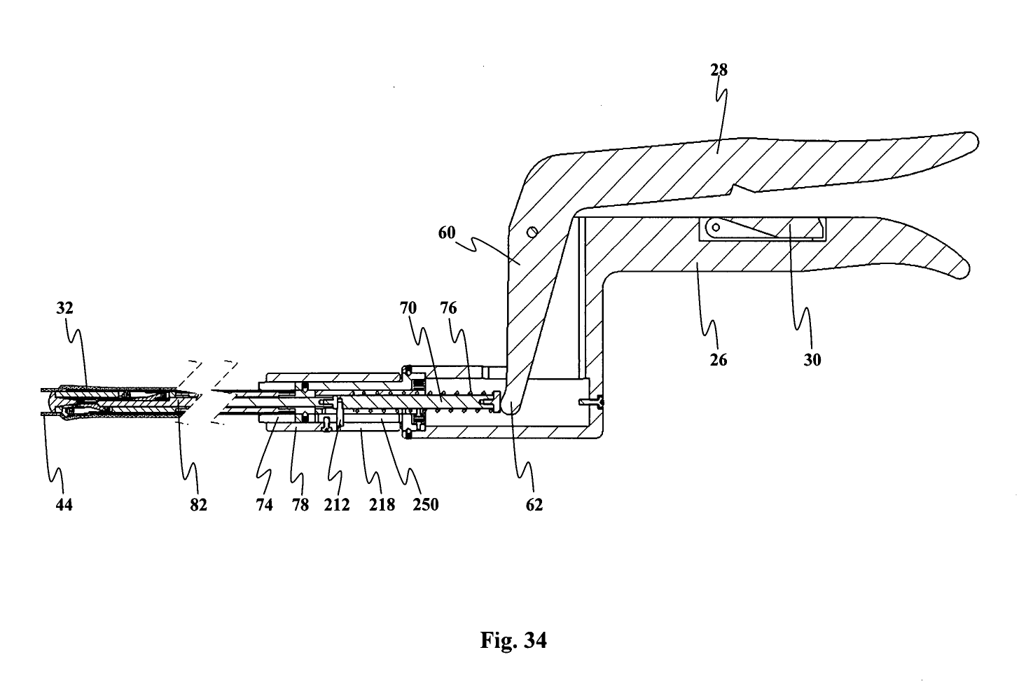

FIG. 34 depicts a situation when the barrel portion with the graft is brought into a vessel, e.g., a femoral artery 32, in accordance with some embodiments of the invention. The latch may be removed and the handles may be pressed such that the lever arm 60 urges the spring guide shaft 70 against the biasing spring 76 to protract distally together with the pushing rod 82. During protraction, the guiding pin 212 preferably slides in a distal direction along slot 218 made in the external guiding bushing 78 and along slot 250 made in the collet member. When the handles are brought closer to the extent shown in FIG. 34, the recessed zones of the pushing rod passes along the rear groups of the wings and levers without engaging them. When the pushing rod approaches the frontal groups of the wings, the non-recessed zones of the forward head approach them and cause them to pivot. This may be associated with tensioning of the graft wall as it has been explained above with reference to FIG. 24.

It should be noted that the length of the slot 218 may be selected in such a manner that when the wings have been pivoted, the guiding pin 212 still does not approach the distal end of the slot.

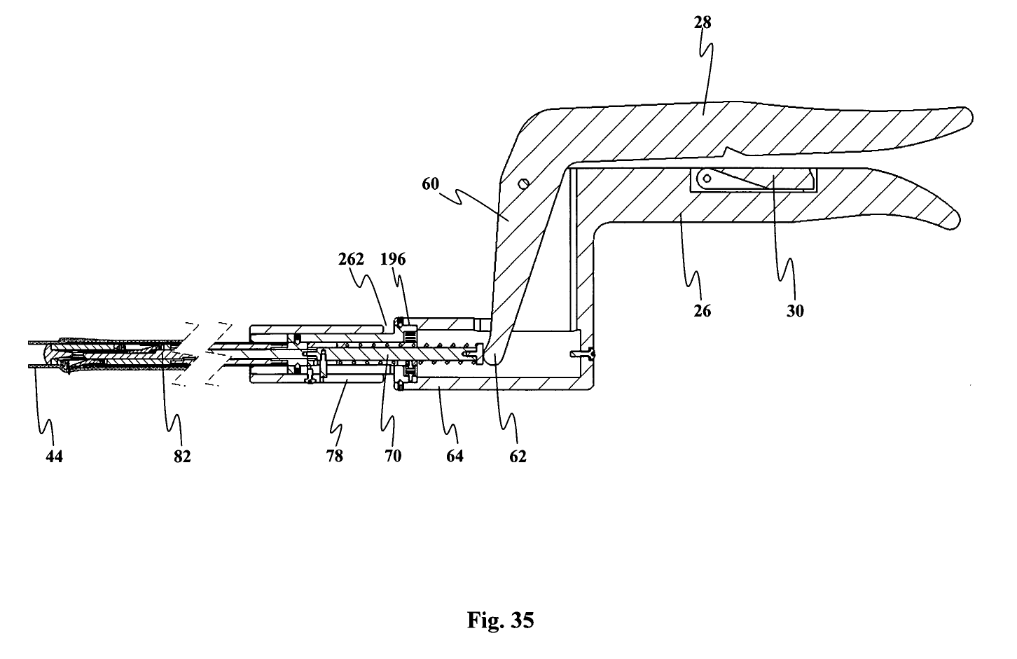

FIG. 35 shows a step when the handles were brought closer such that they almost approached each other, in accordance with some embodiments of the invention. This is associated with additional protracting of the spring guide shaft 70 and of the pushing rod 82 in a distal direction. At the end of the protracting stroke, the pushing rod preferably engages and preferably actuates the levers of the frontal groups, as explained with reference to FIG. 24: i.e., it pushes a plurality of the frontal staples to protrude out of the working portion such that the staples penetrate the graft wall and the vessel wall and reliably affix the distal end of the graft to the distal end of the vessel. It will also be appreciated that the length of the slot 218 may be deliberately selected in such a manner that when the guide shaft 70 has been protracted distally, the guiding pin 212 approaches distal end of the slot 218 and eventually pushes external bushing 78 in a distal direction until its lip portion escapes from the annular groove of the collet number. FIG. 35 shows that after the external bushing has been displaced distally, a gap 262 is provided between the end of the external bushing and the forward end 196 of the housing 64.

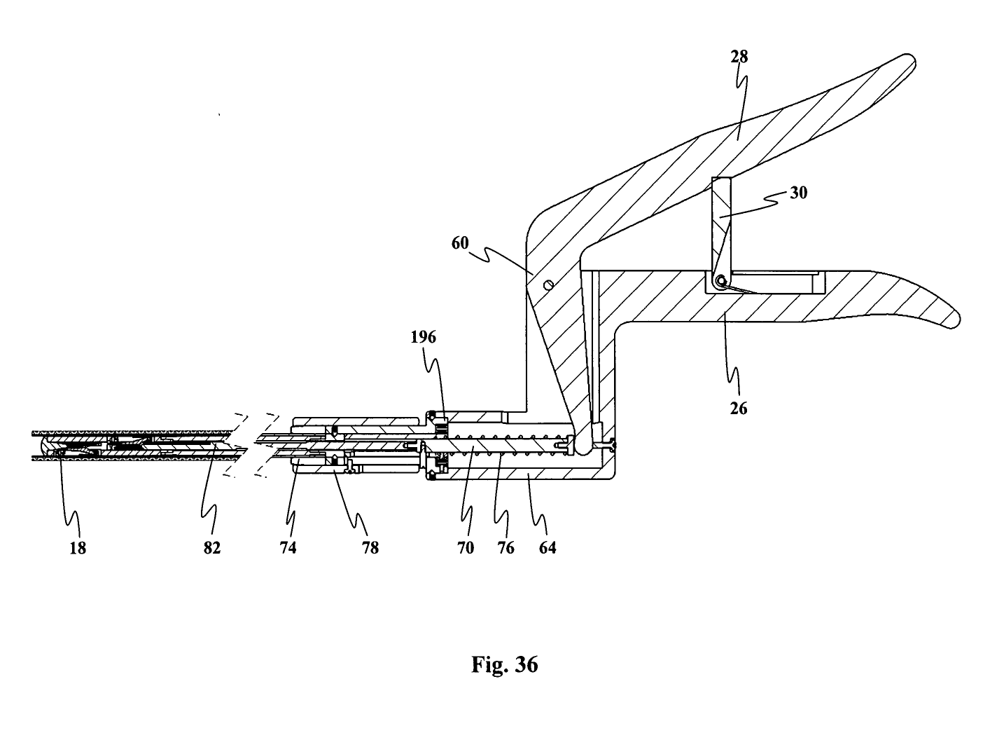

One of skilled in the art will appreciate that, in accordance with some embodiments, when the lip portion does not exert pressure on the leaves of the collet member, the proximal end of the graft is not clamped by the collet member; and, therefore, the graft is no longer connected to the handle portion. Therefore, the barrel portion may be displaced proximally along the graft so as to bring the working portion to that location where the proximal end of the graft should be affixed. FIG. 36 shows the situation when the external bushing 78 has been pushed by the pushing pin distally, and there is no connection between the handle portion and the proximal end 20 of the graft.

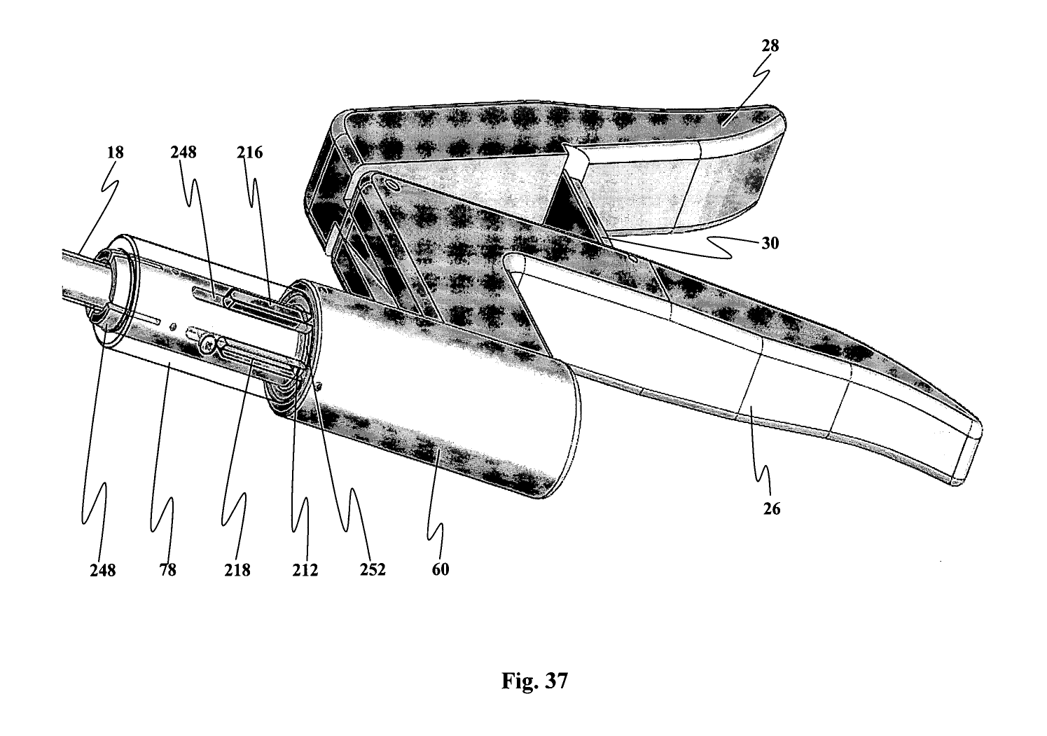

FIG. 37 shows the situation when the barrel portion and the working portion have been brought to a location where the proximal end of the graft should be affixed, in accordance with some embodiments of the invention. At that point, the operator releases handles 26, 28 to allow the biasing spring 76 to forcibly retract the shaft 70 together with the pushing rod 82 proximally. In this position, the handles of the handle portion may be locked by the latch 30 swiveled in the upright position. Now torsion spring 196 urges the shaft 70 to rotate clockwise along with the inner guiding bushing and the pushing rod 82. The rotation movement is allowed since the gap 262 is provided between the external bushing and the housing 60, and thus, the guiding pin 212 is free to slide along the transversal slot made in the collet member. The length of the transversal slot 252 may be deliberately selected in such a manner that the pin slides circumferentially along the slot 252 from a first position, in which it has been aligned with the longitudinal slot 218, into a second position, in which it will be now aligned with the longitudinal slot 216. As explained above with reference to FIG. 30 and FIG. 31, the longitudinal slots may be circumferentially separated from one another by about 60 degrees (for example), and thus, the pushing rod will be turned circumferentially by the torsion spring exactly to the same extent. Since the rear groups of the wings and levers are also circumferentially separated from the frontal groups by about 60 degrees (for example), the non-recessed zones of the forward head may now engage and actuate the rear groups of the wings and levers if the pushing rod is protracted distally.

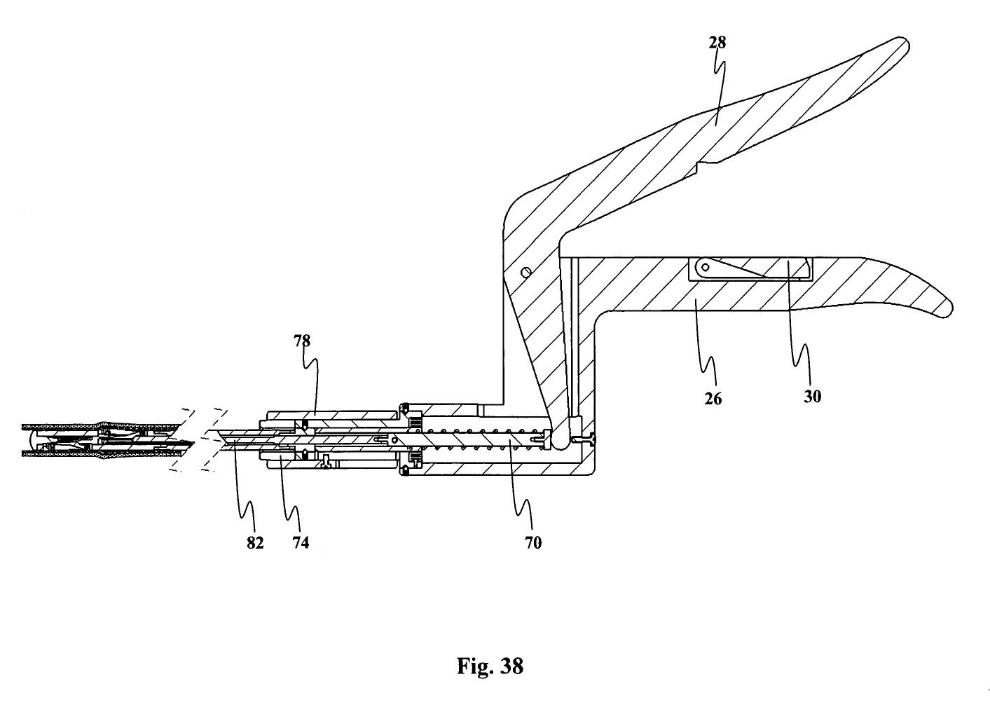

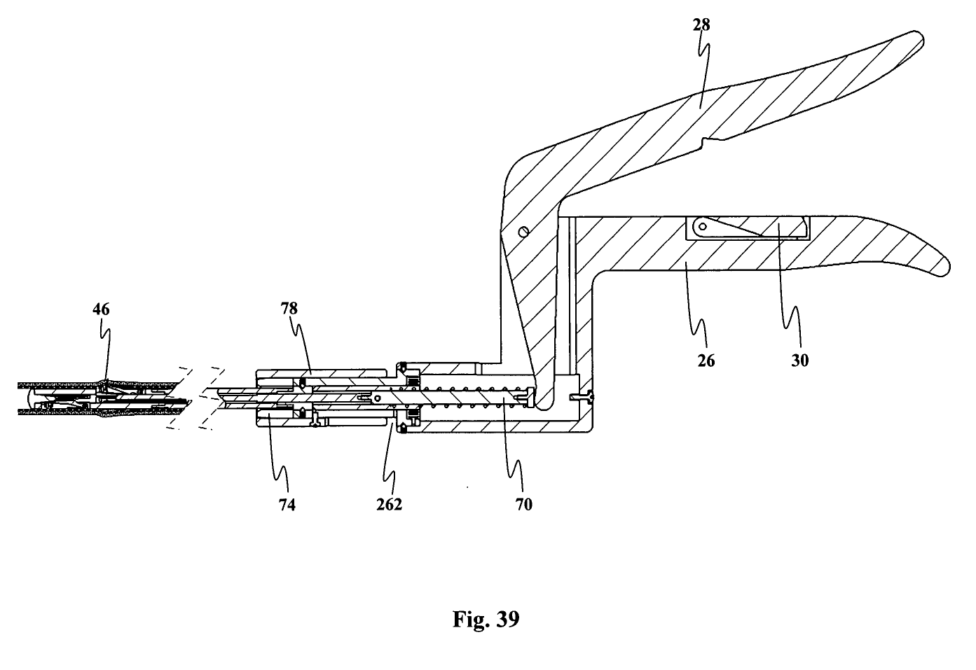

FIG. 38 and FIG. 39 show a device of the invention during affixing the proximal end of the graft, in accordance with some embodiments of the invention. At this stage, the latch 28 may be released and swiveled in a horizontal position such that the operator can press the handles 26,28 to bring them close. Pressing the handles may be associated with protracting the spring shaft 70 and the pushing rod 82 towards the rear groups of wings and levers. The spring shaft 70 is then free to protract distally since the guiding pin can now slide along the longitudinal slot 216 of the external bushing 78 and along longitudinal slot 248 of the collet member. Once the non-recessed zones of the pushing rod approach the rear groups of wings and levers, they engage them and pivot the rear wings similarly to what has been explained above with reference to FIG. 24 and FIG. 34. The further protracting of the pushing rod may be associated with pivoting of the rear levers and affixing the graft by a plurality of rear staples 46 as seen in FIG. 39, as has been explained with reference to FIG. 25 and FIG. 35.

Thus, by virtue of at least some of the embodiments of the present invention, it is possible for a single device to quickly and reliably affix two ends of a stent to two ends of a vessel.

By virtue of at least some embodiments of the present invention, it is possible to affix two opposite ends of a graft to two ends of a vessel. This is possible by bringing the working head to the required location in the course of a simple surgical operation, which does not require opening cavities surrounding the vessel.

It will be appreciated that features disclosed in the foregoing description, and/or in the foregoing drawings and/or following claims both separately and in any combination thereof, are non-limiting and that the present invention may be realized in diverse forms.

When used in the following claims, the terms “comprise,” “include,” “have,” and their conjugates mean “including but not limited to.”

As used herein, the terms “stent”, “graft” or “stent-graft” refer to an intraluminal medical accessory serving as a prosthesis intended to be placed and secured in a body of a human or an animal patient.

As used herein, the term “vessel” refers to any hollow vessels or ducts or cavities available in a mammal body. Non-limiting examples of such passages are arteries, veins, intestines, etc.

As used herein the term “staple” refers to an example of a surgical fastener, a fastener being capable of affixing a prosthesis to a vessel.

As used herein, the term “affixing” refers to the physical attachment of one object to another.

As used herein the term “pushing rod” refers to an example of an extender, an extender being capable of expanding, spreading or stretching forth.

The scope of the invention is defined by the appended claims.

What is claimed is:

1. A device for affixing a prosthesis to a first end and to a second end of a vessel, said device comprising a working portion and a barrel portion, wherein said working portion comprises at least one fastener, wherein said barrel portion is adapted to carry the prosthesis thereon and wherein said device is provided with an extender which is displaceable along the barrel portion so as to cause protrusion of a fastener.

2. The device of claim 1, in which the first end of the prosthesis covers the working portion and is detachably connected thereto and the second end of the prosthesis is detachably connected at a handle portion, wherein the barrel portion is intermediate between the working and handle portions.

3. The device of claim 2, in which the handle portion comprises an upper handle and a lower handle, said handles can be either brought close to each other or released such that the extender can be longitudinally displaced along the barrel portion.

4. The device of claim 1, in which said working portion comprises a main body portion which accommodates therein a plurality of pivoting elements and slotted elements, wherein said pivoting elements and said slotted elements are arranged in frontal groups and in rear groups.

5. The device of claim 4, in which each frontal group and each rear group includes similar combination of elements consisting of a pivoting wing, a pivoting lever and a slotted element.

6. The device of claim 5, in which the slotted elements of the frontal groups are loaded with fasteners being a first plurality of staples and the slotted elements of the rear groups are loaded with fasteners being a second plurality of staples.

7. The device of claim 4, in which said frontal groups and said rear groups are symmetrically arranged along a circumference of the main body portion.

8. The device of claim 7, in which said main body portion accommodates three frontal groups and three rear groups.

9. The device of claim 8, in which each frontal group is separated from an adjacent frontal group by an interval of 120 degrees and each rear group is separated from an adjacent rear group by an interval of 120 degrees.

10. The device of claim 4, in which none of the rear groups is in alignment with any of the frontal groups.

11. The device of claim 4, in which one end of said main body portion is detachably closed by a plug and the opposite end of the body portion is detachably connected to the barrel portion.

12. The device of claim 4, in which said frontal groups and said rear groups reside in respective cut outs made in the main body portion.

13. The device of claim 4, in which said barrel portion comprises a tubular barrel with the extender extending along the barrel, wherein the extender is a pushing rod, and the pushing rod is provided with a forward head, with a shaft portion and with a rear end.

14. The device of claim 13, in which upon longitudinal displacement of the pushing rod the forward head thereof is protracted within the main body portion of the working portion or is retracted therefrom.

15. The device of claim 14, in which upon protracting of the forward head within the main body portion either the frontal groups or the rear groups are actuated and cause protrusion of fasteners being a first plurality of staples or fasteners being a second plurality of staples.

16. The device of claim 6, in which pivoting wing of each of the frontal groups or the rear groups is mounted with possibility to pivot with respect to the main body portion and each pivoting lever is mounted within a slot made in the respective pivoting wing such that the pivoting lever is capable to pivot with respect to the pivoting wing.

17. The device of claim 16, in which a butt end of each pivoting wing is provided with a cut out terminating by a flaring exit and a staple is loaded in the cut out.

18. The device of claim 17, in which the slotted element is secured at the butt end of the pivoting wing, such that the staple is retained between the slotted element and the butt end wherein the staple can be forcibly displaced from the cut out through the flaring exit.

19. The device of claim 18, in which said flaring exit is configured and dimensioned to bend the staple when it passes the flaring exit.

20. The device of claim 18, in which each pivoting lever is provided with a front end, which upon pivoting of the lever urges the staple to displace from the cut out through the flaring exit.

21. The device of claim 13, in which said shaft portion is provided with regions having a small diameter and with regions having a large diameter and said forward head is provided with recessed zones interspersing with non recessed zones.

22. The device of claim 21, in which said recessed zones and said non recessed zones are symmetrically arranged along a circumference of the forward head and separated by an interval of 60 degrees.

23. The device of claim 2, in which said handle portion comprises a tubular housing connected to an upper handle, said upper handle being provided with a lever arm, and said handle portion further comprising a spring guide shaft extending along the tubular housing and carrying thereon a biasing spring, the arrangement being such that upon bringing the upper handle close to the lower handle said lever arm is forcibly pivoted against the biasing spring and causes protracting the spring guide shaft along the housing while upon release of the upper handle and the lower handle said spring causes retracting the spring guide shaft and pivoting the lever arm.

24. The device of claim 23, in which said handle portion comprises an inner guiding bushing, a collet member and an external guiding bushing, wherein the rear end of the extender extends along the inner guiding bushing and wherein the extender is a pushing rod.

25. The device of claim 24, in which the rear end of the pushing rod is connected to a forward end of the spring guide shaft, such that the pushing rod and the spring guide shaft can be protracted and retracted simultaneously.

26. The device of claim 24, in which said handle portion is provided with a torsion spring received within the tubular housing, wherein one end of the torsion spring is connected to the spring guide shaft and the other end of the torsion spring is connected to the housing, the arrangement being such that said spring guide shaft is rotatable by the torsion spring

27. The device of claim 26, in which said external guiding bushing and said collet member are provided with two parallel longitudinal slots and said handle portion is provided with a guiding pin which one end is connected to the pushing rod and the opposite end is displaceable along the slots upon protracting or retracting of the pushing rod.

28. The device of claim 27, in which the longitudinal slots of the collet member are connected by a transversal slot and said guiding pin is displaceable along the transversal slot upon rotation of the pushing rod.

29. The device of claim 28, in which said two parallel longitudinal slots of the external guiding bushing and of the collet member are circumferentially separated by 60 degrees.

30. The device of claim 29, in which the rear end of the prosthesis is fastened between the collet member and the external guiding bushing.

31. The device of claim 30, in which said external guiding bushing is displaceable in a distal direction along the collet member upon protracting of the pushing rod.

32. The device of claim 1, in which said vessel is selected from the group consisting of a duct, an artery, a vein, an intestine.

33. The device of claim 1, in which said vessel is a femoral artery.

34. The device of claim 1, in which said prosthesis is a graft.

35. A method for affixing a distal end and a proximal end of a prosthesis to a distal end and a proximal end of a vessel said method comprising:
providing a device which comprises a working portion with at least one fastener;
bringing the working portion to the distal end of the vessel;
affixing the distal end of the prosthesis to the distal end of the vessel by a fastener;
bringing the working portion to the proximal end of the prosthesis;
affixing the proximal end of the prosthesis to the proximal end of the vessel by a fastener; and
evacuating the working portion from the vessel.

36. The method of claim 35, in which said prosthesis is a graft.

37. The method of claim 35, in which said vessel is selected from the group consisting of a duct, an artery, a vein and an intestine.

38. The method of claim 37, in which said vessel is a femoral artery.

39. A method for affixing a prosthesis to a first end and to a second end of a vessel, said method comprising:
providing a device which comprises a rear handle portion, a frontal working portion and an intermediate barrel portion, wherein said frontal working portion is loaded with a first plurality of fasteners and with a second plurality of fasteners, wherein said barrel portion is adapted to carry the prosthesis thereon such that a distal end of the prosthesis is connected to the working portion and a proximal end of the prosthesis is connected at the handle portion and wherein said device is provided with an extender which is displaceable along the barrel portion so as to actuate the frontal working portion;
entering the frontal working portion and at least a portion of the barrel portion within the vessel so as to bring the distal end of the prosthesis to the first end of the vessel;
protracting the extender along the barrel portion so as to actuate the frontal working portion such that the first plurality of fasteners is urged to protrude in a radial direction to affix the distal end of the prosthesis to the first end of the vessel;
disconnecting the proximal end of the prosthesis from the handle portion;
retracting the extender;
retracting the frontal working portion and the barrel portion along the vessel so as to bring the proximal end of the prosthesis to the second end of the vessel;
protracting the extender along the barrel portion so as to actuate the frontal working portion such that the second plurality of fasteners is urged to protrude in the radial direction to affix the proximal end of the prosthesis to the second end of the vessel; and
evacuating the working portion and the barrel portion from the vessel.

40. The method of claim 39, in which said vessel is selected from the group consisting of a duct, an artery, a vein and an intestine.

41. The method of claim 40, in which said vessel is a femoral artery.

42. The method of claim 39, in which said prosthesis is a graft.

43. The method of claim 39, in which said extender is a pushing rod.

44. The method of claim 39, in which said fastener is a staple.
